(19) United States
(12) Patent Application Publication
(10) Pub. No.: US 2015/0148644 A1
(43) Pub. Date: May 28, 2015

(54) NEURO-PROBE DEVICE, IMPLANTABLE ELECTRONIC DEVICE AND METHOD OF FORMING A NEURO-PROBE DEVICE

(71) Applicant: AGENCY FOR SCIENCE, TECHNOLOGY AND RESEARCH, Singapore (SG)

(72) Inventors: Kripesh Vaidyanathan, Singapore (SG); Ruiqi Lim, Singapore (SG); Riyas Katayan Fazalul Rahuman, Singapore (SG); Woo Tae Park, Singapore (SG); Anupama Vijay Govindarajan, Singapore (SG); Minkyu Je, Singapore (SG)

(21) Appl. No.: 14/399,934

(22) PCT Filed: May 7, 2013
(86) PCT No.: PCT/SG2013/000180

§ 371 Date: Nov. 7, 2014

(30) Foreign Application Priority Data
May 7, 2012 (SG) 201203330-4

(51) Int. Cl. A61B 5/04; C03C 4/00; C03B 19/02; A61M 37/00; A61N 1/05

(52) U.S. Cl. CPC A61B 5/04001; A61M 37/00; A61N 1/0551; C03B 19/02; C03C 4/0014; A61M 2210/0693; A61M 2207/00; A61M 2205/04
USPC 600/377; 604/264; 607/116; 65/66

(57) ABSTRACT

A neuro-probe device is provided. The neuro-probe device includes a carrier including bio-resorbable glass, and a neuro-probe mounted on the carrier.

### CROSS-REFERENCE TO RELATED APPLICATION

This application claims the benefit of priority of Singapore Patent Application No. 201203330-4, filed 7 May 2012, the contents of which being hereby incorporated by reference in its entirety for all purposes.

### TECHNICAL FIELD

Various embodiments relate generally to a neuro-probe device, an implantable electronic device for neural recording and/or stimulation and/or drug delivery, and a method of forming a neuro-probe device.

### BACKGROUND

Neuro probes have been used for studying and understanding the function of the brain. The probes can measure and record the neuron action potentials and can be used to stimulate specific brain region to allow more in-depth understanding on the neurons characteristics such as the population encoding, network connectivity and nervous system behavior.

The selection of materials for neuro therapeutic applications depends on various factors like bio-inert and toxicity. Generally, materials such as metal wires and silicon are selected as the materials for the neuro probes applications. One of the major challenges is the compatibility of the probes with the movement of the brain tissue. As the materials used for the probes have a much higher mechanical hardness as compared to the brain matter, the probes may not be compatible with the brain tissue movements. Consequently, the probes may damage the surrounding brain tissue which may lead to more complications.

Neural probes of soft materials like parylene, polymide, SU-8, and materials of switchable stiffness are closer in Young's modulus to the brain. Insertion of the probes with soft materials may require separate insertion devices that could leave a much larger footprint than the probe device, thus damaging the brain tissue.

### SUMMARY

According to one embodiment, a neuro-probe device is provided. The neuro-probe device includes a carrier including bio-resorbable glass, and a neuro-probe mounted on the carrier.

According to one embodiment, an implantable electronic device for neural recording and/or stimulation and/or drug delivery is provided. The implantable electronic device includes at least one neuro-probe device.

According to one embodiment, a method of forming a neuro-probe device is provided. The method includes forming a carrier comprising bio-resorbable glass, and mounting a neuro-probe to the carrier.

### BRIEF DESCRIPTION OF THE DRAWINGS

In the drawings, like reference characters generally refer to the same parts throughout the different views. The drawings are not necessarily to scale, emphasis instead generally being placed upon illustrating the principles of the invention. In the following description, various embodiments of the invention are described with reference to the following drawings, in which:

FIG. 1 shows a schematic diagram of a neuro-probe device according to one embodiment.

FIG. 2a shows a three-dimensional view of a neuro-probe device according to one embodiment.

FIG. 2b shows a cross-sectional view of a neuro-probe device according to one embodiment.

FIGS. 2c and 2d show a degradation of a bio-resorbable glass of a carrier of a neuro-probe device according to one embodiment.

FIG. 3 shows a schematic diagram of a neuro-probe device according to one embodiment.

FIG. 4 shows a schematic diagram of a neuro-probe device according to one embodiment.

FIG. 5a shows a three-dimensional view of a neuro-probe device according to one embodiment.

FIG. 5b shows a cross-sectional view of a neuro-probe device according to one embodiment.

FIGS. 5c-5f show a degradation of a bio-resorbable glass of a carrier of a neuro-probe device according to one embodiment.

FIG. 6a shows a three-dimensional view of a neuro-probe device according to one embodiment.

FIG. 6b shows a cross-sectional view of a neuro-probe device according to one embodiment.

FIGS. 6c-6f show a degradation of a neuro-probe device according to one embodiment.

FIG. 7a shows a three-dimensional view of a neuro-probe device according to one embodiment.

FIG. 7b shows a cross-sectional view of a neuro-probe device according to one embodiment.

FIGS. 7c-7e show a degradation of a bio-resorbable glass of a carrier of a neuro-probe device according to one embodiment.

FIG. 8 shows a schematic diagram of an implantable electronic device for neural recording and/or stimulation and/or drug delivery according to one embodiment.

FIG. 9 shows a flowchart of a process of forming a neuro-probe device according to one embodiment.

FIGS. 10a-10f show a process of forming a neuro-probe device according to one embodiment.

FIG. 11 shows a schematic diagram of a wafer used as a mold for forming a neuro-probe device according to one embodiment.

FIG. 12 shows a schematic diagram of a glass wafer having a plurality of carriers for forming a neuro-probe device according to one embodiment.

FIG. 12b shows a side view of a carrier of a neuro-probe device according to one embodiment.

FIGS. 13a-13h show a process of forming a neuro-probe device according to one embodiment.

FIGS. 14a-14h show a process of forming a neuro-probe device according to one embodiment.

FIGS. 15a-15m show a process of forming a neuro-probe device according to one embodiment.

FIG. 16 shows five bio-resoluble glass materials used in an experiment according to one embodiment.

FIG. 17 shows a graph of weight loss (%) plotted against time (hour) for five bio-resorbable glass materials in deionized water according to one embodiment.

FIG. 18 shows an experimental setup of a degradation test in simulated brain fluid for two bio-resoluble glass materials according to one embodiment.

FIG. 19 shows a graph of weight loss (%) plotted against time (hour) for two bio-resoluble glass materials in simulated brain fluid according to one embodiment.

FIG. 20 shows a fluoroscope image of five bio-resoluble glass materials on a pig specimen according to one embodiment.

FIG. 21a shows a fluoroscope image of five bio-resoluble glass materials about 4 hours after implantation according to one embodiment.

FIG. 21b shows a fluoroscope image of five bio-resoluble glass materials on the next day of the implantation according to one embodiment.

FIG. 22a shows microscope images of a tissue segment of an implanted bio-resoluble glass according to one embodiment.

FIG. 22b shows microscope images of a tissue segment of an implanted bio-resoluble glass according to one embodiment.

FIG. 23 shows a graph of weight loss (%) plotted against time (hour) for a sample according to one embodiment.

FIG. 24 shows a graph of weight loss (%) plotted against time (minute) for two samples according to one embodiment.

FIG. 25 shows an in vivo degradation test of three samples in a pig specimen according to one embodiment.

FIG. 26a shows a fluoroscope image of three samples after implantation according to one embodiment.

FIG. 26b shows a fluoroscope image of three samples one day after implantation according to one embodiment.

FIG. 26c shows a fluoroscope image of three samples two days after implantation according to one embodiment.

FIG. 26d shows a fluoroscope image of three samples three days after implantation according to one embodiment.

### DETAILED DESCRIPTION

Embodiments of a neuro-probe device, an implantable electronic device for neural recording and/or stimulation and/or drug delivery, and a method of forming a neuro-probe device will be described in detail below with reference to the accompanying figures. It will be appreciated that the embodiments described below can be modified in various aspects without changing the essence of the invention.

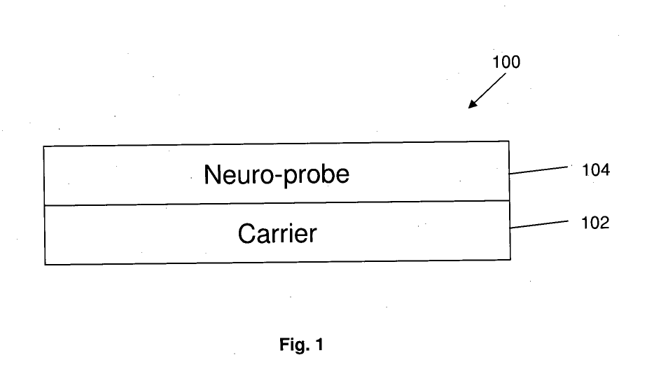

FIG. 1 shows a schematic diagram of a neuro-probe device 100. The neuro-probe device 100 includes a carrier 102. The neuro-probe device 100 also includes a neuro-probe 104 mounted on the carrier 102. The carrier 102 includes bio-resorbable glass.

In one embodiment, the neuro-probe may include a polymer layer disposed above the carrier and an electrode layer disposed above the polymer layer. The bio-resorbable glass material may have a single degradation rate.

In one embodiment, the carrier may include at least one recess formed in a surface of the carrier facing the polymer layer. The neuro-probe device 100 may further include drug and/or chemical disposed in the at least one recess of the carrier.

In one embodiment, the polymer layer may include at least one cavity. The neuro-probe device 100 may further include drug and/or chemical disposed in the at least one cavity of the polymer layer.

In one embodiment, the carrier may include a plurality of sections. The sections of the carrier may include different bio-resorbable glass materials.

In one embodiment, the carrier may include a recess formed in a surface of each section of the carrier facing the polymer layer. The neuro-probe device 100 may further include drug and/or chemical disposed in each recess of the carrier.

In one embodiment, the polymer layer may include a plurality of cavities. Each cavity of the polymer layer may be formed above a corresponding section of the carrier. The neuro-probe device 100 may further include drug and/or chemical disposed in each cavity of the polymer layer.

In one embodiment, the carrier may include a planar portion having a first surface and a second surface facing away from the first surface. Two opposite sides of the first surface and two opposite sides of the second surface may converge to form a tip.

In one embodiment, the bio-resorbable glass may include but is not limited to fluoride phosphate based soluble glass, zinc phosphate based soluble glass, copper phosphate based soluble glass, boron trioxide based soluble glass, and bioactive glass. The electrode layer may include but is not limited to conductive materials. The polymer layer may include but is not limited to parylene, polyimide, polydimethylsiloxane (PDMS) and SU-8. The drug and/or chemical may include but is not limited to maltose with drug.

Different configurations of the neuro-probe device 100 can be used. Different configurations of the neuro-probe device 100 can include different types of the carrier 102 and different types of the neuro-probe 104.

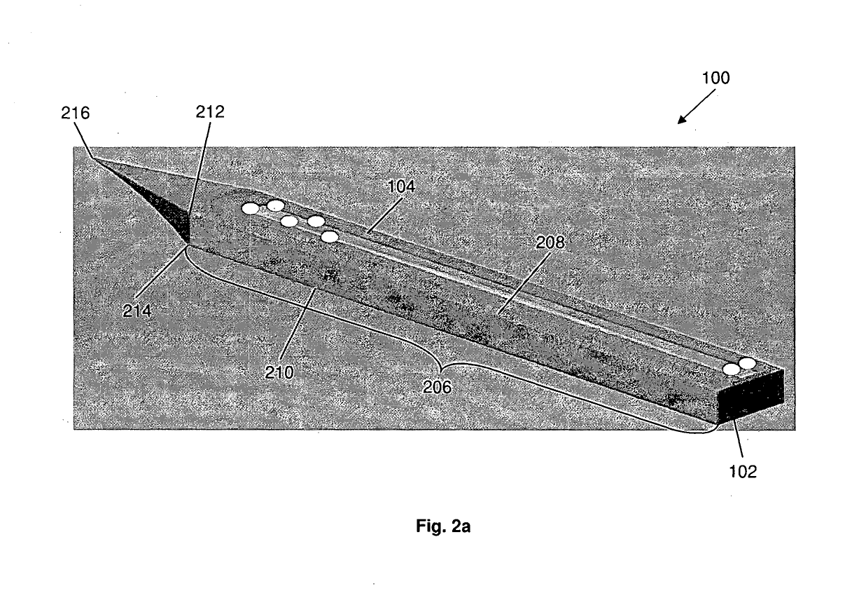

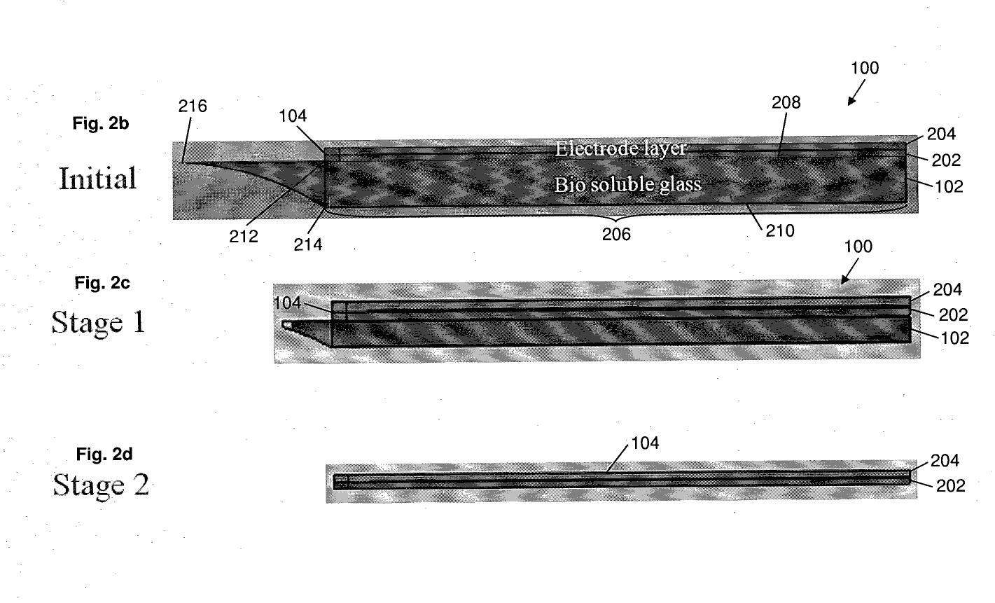

FIG. 2a shows a three-dimensional view of an exemplary neuro-probe device 100. FIG. 2b shows a cross-sectional view of the exemplary neuro-probe device 100. In one embodiment, the neuro-probe 104 of the neuro-probe device 100 has a polymer layer 202 disposed above the carrier 102. The neuro-probe 104 also has an electrode layer 204 disposed above the polymer layer 202. The neuro-probe 104 may be a flexible neuro-probe that is configured to be implantable into a biological tissue.

In one embodiment, the polymer layer 202 includes but is not limited to parylene, polyimide, polydimethylsiloxane (PDMS) and SU-8. The electrode layer 204 includes conductive materials. The conductive materials may include but are not limited to gold.

The carrier 102 of the neuro-probe device 100 has a planar portion 206 having a first surface 208 and a second surface 210 facing away from the first surface 208. Two opposite sides (only one side 212 is shown) of the first surface 208 and two opposite sides (only one side 214 is shown) of the second surface 210 converge to form a tip 216.

The bio-resorbable glass material used for the carrier 102 has a single degradation. After the neuro-probe device 100 is inserted into the brain tissue, the bio-resorbable glass material may start to degrade when it interacts with the cerebrospinal fluid as shown in FIG. 2c. The bio-resorbable glass material may degrade completely leaving the neuro-probe 104 behind as shown in FIG. 2d.

In one embodiment, the neuro-probe device 100 of FIGS. 2a and 2b may be a single bio-soluble glass probe.

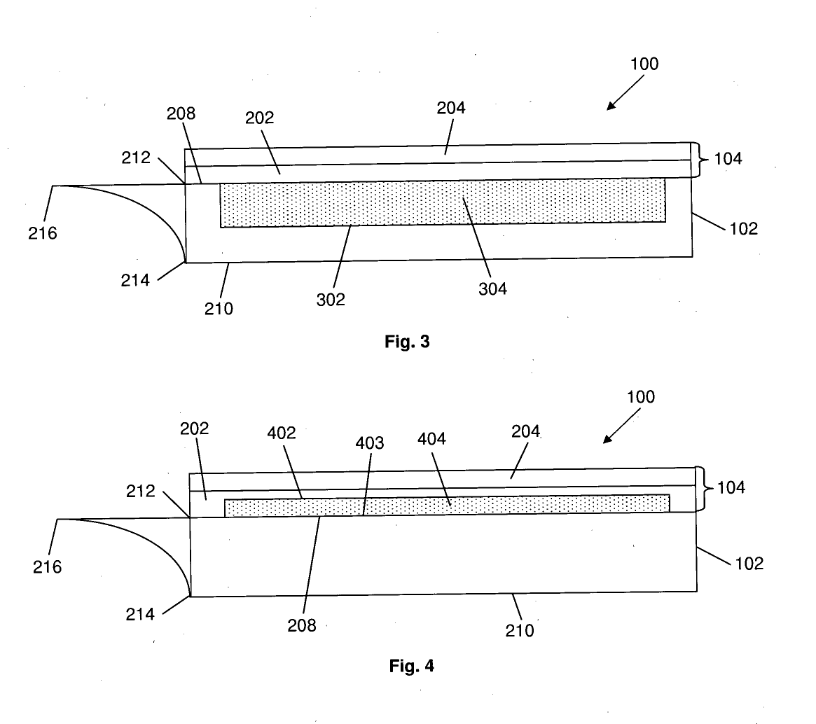

In one embodiment, as shown in FIG. 3, the carrier 102 of the neuro-probe device 100 may have at least one recess 302 formed in a surface of the carrier 102 facing the polymer layer 202 (e.g. the first surface 208 of the carrier 102). For illustration purposes, only one recess 302 is shown in FIG. 3. The number of recesses can vary in different embodiments. Drug and/or chemical 304 may be disposed in the recess 302 of the carrier 102. The drug and/or chemical 304 may include but is not limited to maltose with drug. The recess 302 with the drug and/or chemical 304 may be a drug reservoir of the neuro-probe device 100.

As the bio-resorbable glass material of the carrier 102 degrades in the brain tissue, the drug and/or chemical 304 disposed in the recess 302 of the carrier 102 can be released to the brain neuron. Thus, the neuro-probe device 100 can be used for drug delivery.

Alternatively, as shown in FIG. 4, the polymer layer 202 of the neuro-probe device 100 may have at least one cavity 402. For illustration purposes, only one cavity 402 is shown in FIG. 3. The number of cavities can vary in different embodiments. The cavity 402 may be formed in the surface 403 of the polymer layer 202 facing the carrier 102. Drug and/or chemical 404 may be disposed in the cavity 402 of the polymer layer 202. The drug and/or chemical 404 may include but is not limited to maltose with drug. The cavity 402 with the drug and/or chemical 404 may be a drug reservoir of the neuro-probe device 100. As the bio-resorbable glass material of the carrier 102 degrades in the brain tissue, the drug and/or chemical 404 disposed in cavity 402 of the polymer layer 202 can be released to the brain neuron.

In one embodiment, the neuro-probe device 100 of FIGS. 3 and 4 may be a single bio-soluble glass probe with a drug reservoir.

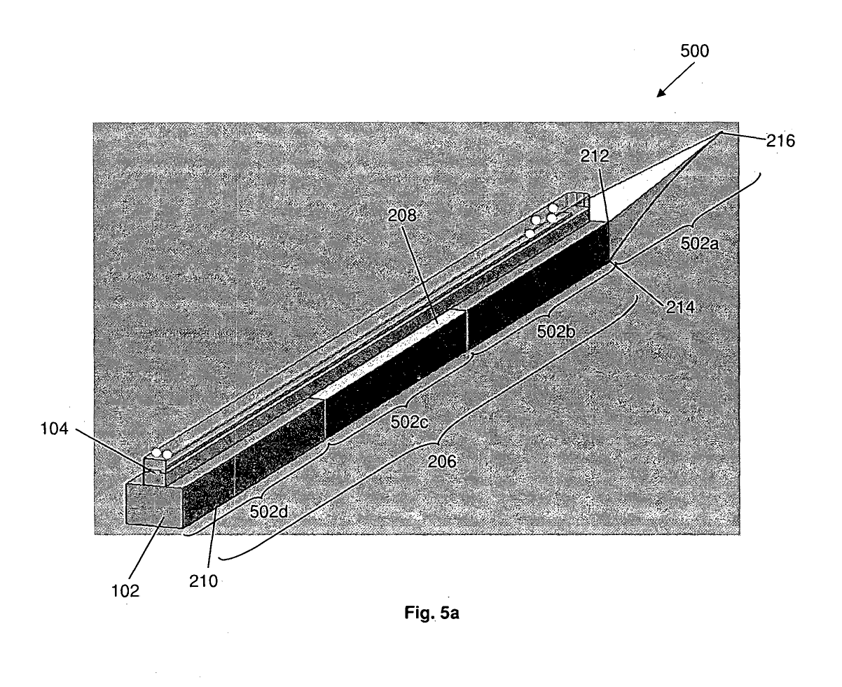

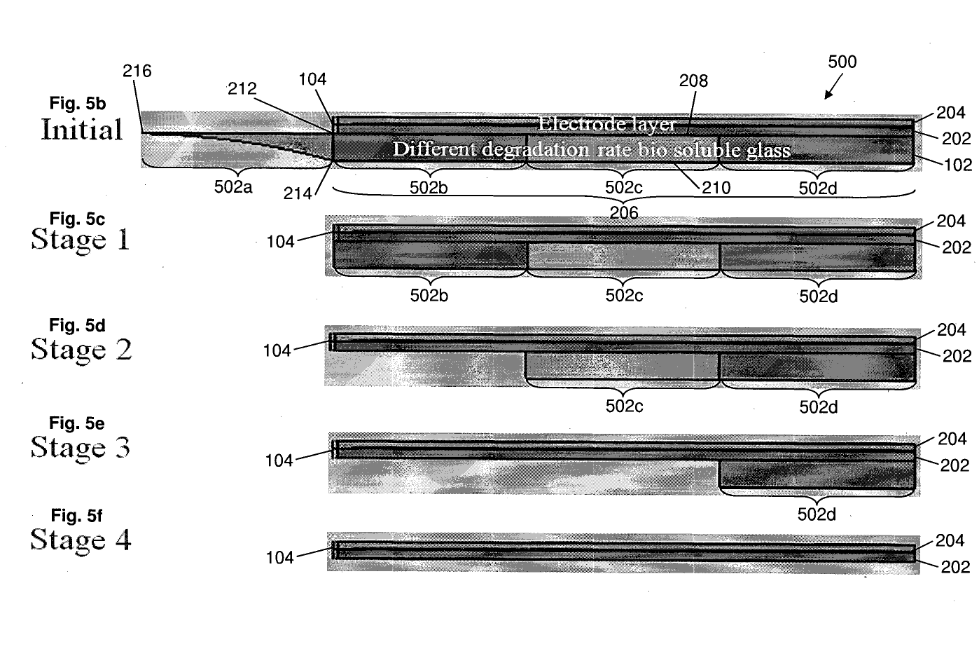

FIG. 5a shows a three dimensional view of another exemplary neuro-probe device 500. FIG. 5b shows a cross-sectional view of the exemplary neuro-probe device 500. In one embodiment, the neuro-probe device 500 has a similar structure as the neuro-probe 100 of FIGS. 2a and 2b, except that the carrier 102 of the neuro-probe device 500 has a plurality of sections (e.g. a first section 502a, a second section 502b, a third section 502c and a fourth section 502d). The first section 502a, the second section 502b, the third section 502c and the fourth section 502d have different bio-resorbable glass materials. As such, the bio-resorbable glass material of each of the first section 502a, the second section 502b, the third section 502c and the fourth section 502d has a different degradation rate.

The number of sections of the carrier 102 can vary in different embodiments. The number of bio-resorbable glass materials used for the carrier 102 can also vary in different embodiments. The number of sections of the carrier 102 may correspond to the number of bio-resorbable glass materials used for the carrier 102.

The bio-resorbable glass material of the first section 502a may have the fastest degradation rate, the bio-resorbable glass material of the second section 502b may have the second fastest degradation rate, the bio-resorbable glass material of the third section 502c may have the third fastest degradation rate, and the bio-resorbable glass material of the fourth section 502d may have the slowest degradation rate. The degradation rate of the bio-resorbable glass materials of the first section 502a, the second section 502b, the third section 502c and the fourth section 502d may be different in other embodiments.

After the neuro-probe device 500 is inserted into the brain tissue, the different bio-resorbable glass materials of the first section 502a, the second section 502b, the third section 502c and the fourth section 502d of the carrier may interact with the cerebrospinal fluid. The bio-resorbable glass material of the first section 502a having the fastest degradation rate may degrade completely first as shown in FIG. 5c. The bio-resorbable glass material of the second section 502b having the second fastest degradation rate may then degrade completely as shown in FIG. 5d. The bio-resorbable glass material of the third section 502c having the third fastest degradation rate may then degrade completely as shown in FIG. 5e. The bio-resorbable glass material of the fourth section 502d having the slowest degradation rate may degrade completely, leaving the neuro-probe 104 behind as shown in FIG. 5f.

In one embodiment, the neuro-probe device 500 may be a multi bio-soluble glass probe.

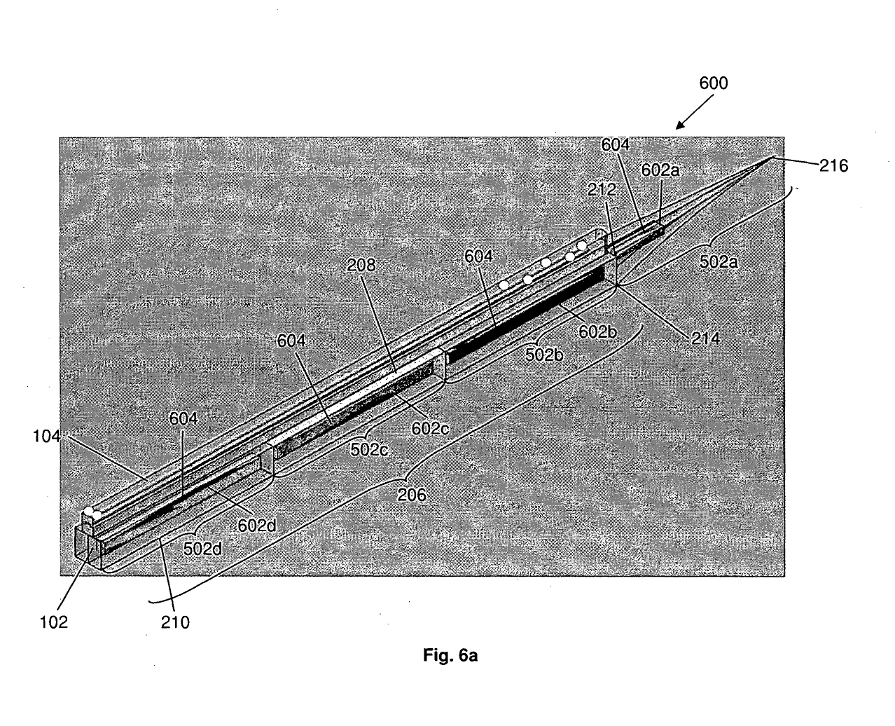

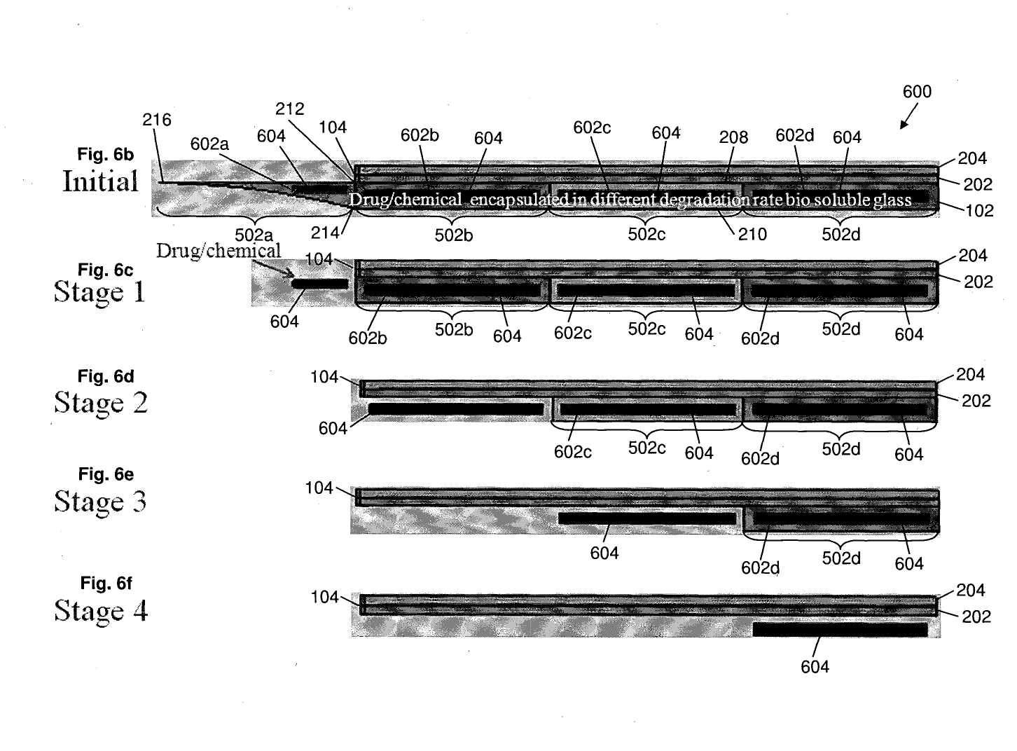

FIG. 6a shows a three dimensional view of another exemplary neuro-probe device 600. FIG. 6b shows a cross-sectional view of the exemplary neuro-probe device 600. In one embodiment, the neuro-probe device 600 has a similar structure as the neuro-probe 500 of FIGS. 5a and 5b, except that the carrier 102 includes a recess (e.g. a first recess 602a, a second recess 602b, a third recess 602c and a fourth recess 602d) formed in a surface of each section 502a-d of the carrier 102 facing the polymer layer 202 (i.e. the first surface 208 of the carrier 102).

Drug and/or chemical 604 may be disposed in each of the first recess 602a, the second recess 602b, the third recess 602c and the fourth recess 602d of the carrier 102. The drug and/or chemical 604 may include but is not limited to maltose with drug. The first recess 602a, the second recess 602b, the third recess 602c and the fourth recess 602d with the drug and/or chemical 304 may be drug reservoirs incorporated into the neuro-probe device 600 (e.g. into the carrier 102 of the neuro-probe device 600).

In one embodiment, the type of drug and/or chemical 604 in the first recess 602a, the second recess 602b, the third recess 602c and the fourth recess 602d may be the same. The volume of drug and/or chemical 604 in the first recess 602a, the second recess 602b, the third recess 602c and the fourth recess 602d may be the same.

In another embodiment, the type of drug and/or chemical 604 in the first recess 602a, the second recess 602b, the third recess 602c and the fourth recess 602d may be different. The volume of drug and/or chemical 604 in the first recess 602a, the second recess 602b, the third recess 602c and the fourth recess 602d may be different.

After the neuro-probe device 600 is inserted into the brain tissue, the different bio-resorbable glass materials of the first section 502a, the second section 502b, the third section 502c and the fourth section 502d of the carrier may interact with the cerebrospinal fluid. As the different bio-resorbable glass materials degrade in the brain tissue, the drug and/or chemical 604 disposed in the first recess 602a, the second recess 602b, the third recess 602c and the fourth recess 602d can be released to the brain neuron. Thus, the neuro-probe device 600 can be used for drug delivery.

The bio-resorbable glass material of the first section 502a having the fastest degradation rate may degrade completely first and the drug and/or chemical 604 disposed in the first recess 602a may be released as shown in FIG. 6c. The bio-resorbable glass material of the second section 502b having the second fastest degradation rate may then degrade completely and the drug and/or chemical 604 disposed in the second recess 602b may be released as shown in FIG. 6d. The bio-resorbable glass material of the third section 502c having the third fastest degradation rate may then degrade completely and the drug and/or chemical 604 disposed in the third recess 602c may be released as shown in FIG. 6e. The bio-resorbable glass material of the fourth section 502d having the slowest degradation rate may degrade completely and the drug and/or chemical 604 disposed in the fourth recess 602d may be released, leaving the neuro-probe 104 behind as shown in FIG. 6f.

In one embodiment, the neuro-probe device 600 may be a multi bio-soluble glass probe with drug reservoir(s). The neuro-probe device 600 can release the drug and/or chemical 604 at different timings/intervals due to the different degradation rates of the different bio-resorbable glass materials of the first section 502a, the second section 502b, the third section 502c and the fourth section 502d of the carrier 102. Releasing the drug and/or chemical 604 at different timings/intervals can help to reactivate the brain neuron.

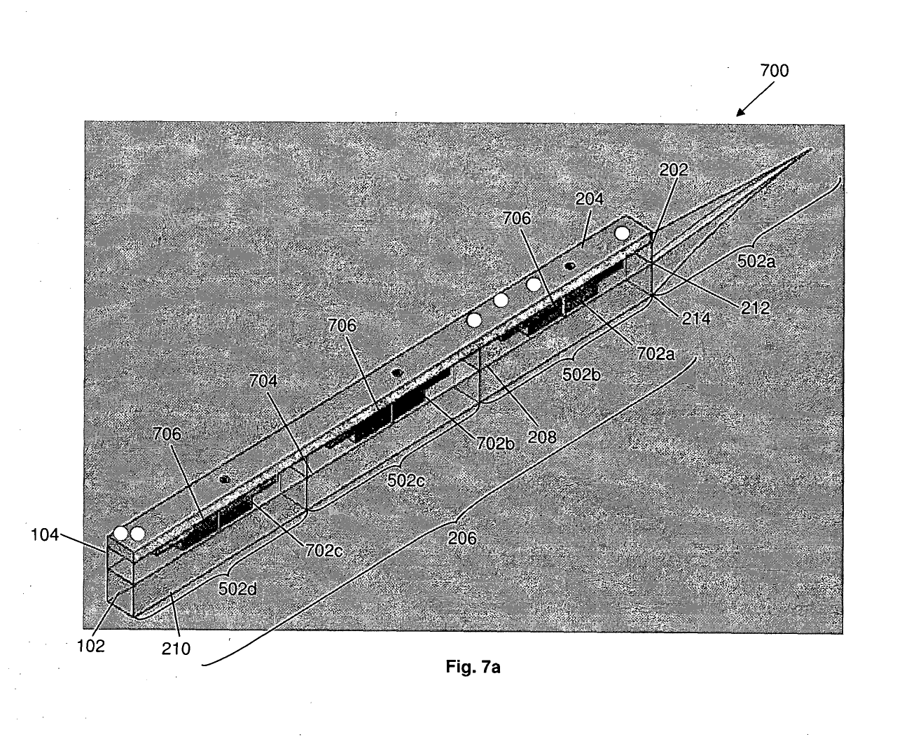

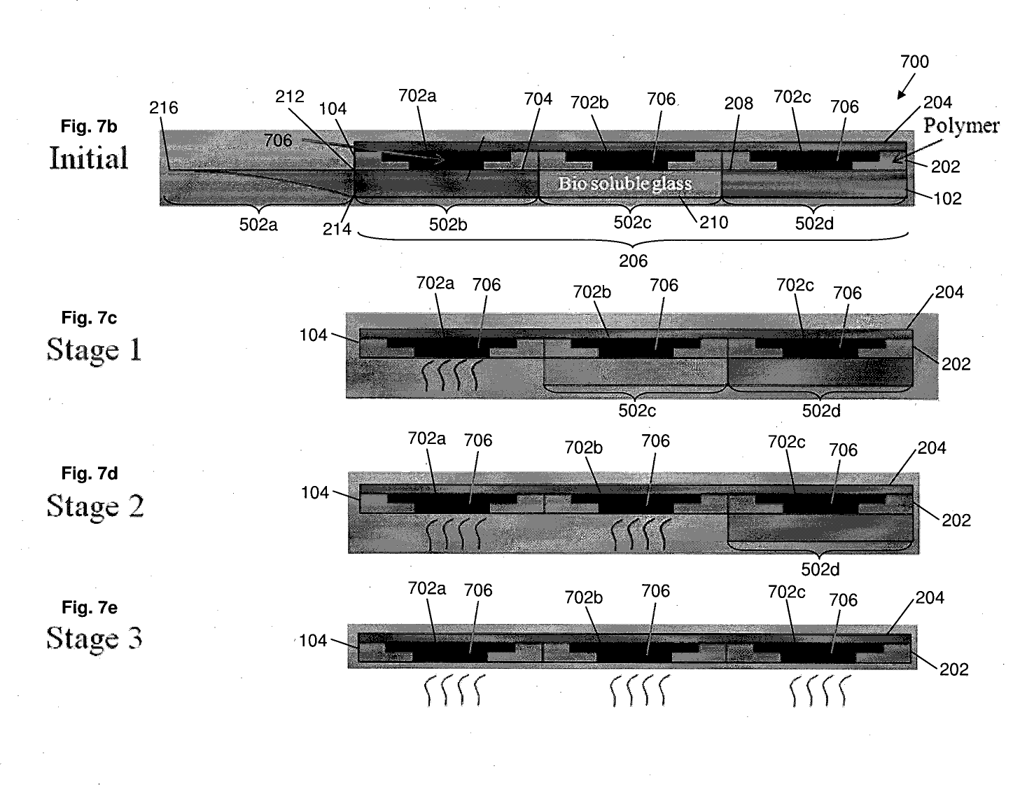

FIG. 7a shows a three dimensional view of another exemplary neuro-probe device 700. FIG. 7b shows a cross-sectional view of the exemplary neuro-probe device 700. In one embodiment, the neuro-probe device 700 has a similar structure as the neuro-probe 500 of FIGS. 5a and 5b, except that the polymer layer 202 includes a plurality of cavities (e.g. a first cavity 702a, a second cavity 702b and a third cavity 702c). Each cavity 702a-c is formed above a corresponding section 502b-d of the carrier 202. The first cavity 702a is formed above the second section 502b, the second cavity 702b is formed above the third section 502c, and the third cavity 702c is formed above the fourth section 502d.

The first cavity 702a, the second cavity 702b and the third cavity 702c may be formed in the surface 704 of the polymer layer 202 facing the carrier 102. Drug and/or chemical 706 may be disposed in the first cavity 702a, the second cavity 702b and the third cavity 702c of the polymer layer 202. The drug and/or chemical 706 may include but is not limited to maltose with drug. The first cavity 702a, the second cavity 702b and the third cavity 702c with the drug and/or chemical 304 may be drug reservoirs incorporated into the neuro-probe device 700 (e.g. into the polymer layer 202 of the neuro-probe device 700).

In one embodiment, the type of drug and/or chemical 706 in the first cavity 702a, the second cavity 702b and the third cavity 702c may be the same. The volume of drug and/or chemical 706 in the first cavity 702a, the second cavity 702b and the third cavity 702c may be the same.

In another embodiment, the type of drug and/or chemical 706 in the first cavity 702a, the second cavity 702b and the third cavity 702c may be different. The volume of drug and/or chemical 706 in the first cavity 702a, the second cavity 702b and the third cavity 702c may be different.

After the neuro-probe device 700 is inserted into the brain tissue, the different bio-resorbable glass materials of the first section 502a, the second section 502b, the third section 502c and the fourth section 502d of the carrier may interact with the cerebrospinal fluid. As the different bio-resorbable glass materials degrade in the brain tissue, the drug and/or chemical 706 disposed in the first cavity 702a, the second cavity 702b and the third cavity 702c can be released to the brain neuron. Thus, the neuro-probe device 700 can be used for drug delivery.

The bio-resorbable glass material of the first section 502a having the fastest degradation rate may degrade completely first, the bio-resorbable glass material of the second section 502b having the second fastest degradation rate may then degrade completely and the drug and/or chemical 706 disposed in the first cavity 702a may be released as shown in FIG. 7c. The bio-resorbable glass material of the third section 502c having the third fastest degradation rate may then degrade completely and the drug and/or chemical 706 disposed in the second cavity 702b may be released as shown in FIG. 7d. The bio-resorbable glass material of the fourth section 502d having the slowest degradation rate may degrade completely and the drug and/or chemical 604 disposed in the third cavity 702c may be released, leaving the neuro-probe 104 behind as shown in FIG. 7e.

In one embodiment, the neuro-probe device 700 may be a multi bio-soluble glass probe with drug reservoir(s). The neuro-probe device 700 can release the drug and/or chemical 706 at different timings due to the different degradation rates of the different bio-resorbable glass materials of the first section 502a, the second section 502b, the third section 502c and the fourth section 502d of the carrier 102. Releasing the drug and/or chemical 706 at different timings/intervals can help to reactivate the brain neuron.

The above described neuro-probe devices have the stiffness for a smooth penetration of the brain tissue as well as the ability to biodegrade after implantation. The biodegradability of the carrier of the neuro-probe devices can prevent tissue damage from occurring as a result of the movement of the brain. Drug delivery can also be incorporated into the carrier or the polymer layer of the neuro-probe to facilitate re-activation of the neurons.

The above described neuro-probe devices can be bio-resorbable (bio-glass) neuro-probes with customizable degradation and drug release by using different bio-resorbable glass with different degradation rates for the carrier and incorporating a drug reservoir in the carrier or in the polymer layer of the neuro-probe. The bioresorbable glass substrate (e.g. carrier) can be customized to degrade at specific timing and releasing the drug to neurons to facilitate treatment or anti-inflammation applications.

The bio-resorbable glass used for the carrier of the neuro-probe devices can be rigid and have high mechanical strength properties that enable a smooth penetration of the brain tissue. The bio-resorbable glass can be biocompatible, biodegradable, and can leave near zero residue after degradation. The bio-resorbable glass can have ease of processing and can be possible to be integrated with other features, e.g. chemical reservoir, optic actuator.

The neuro-probe can be a flexible electrode. The flexible electrode can have a flexible substrate. The neuro-probe can be biocompatible and have high dielectric properties. The neuro-probe can allow the embedding of electrical conductor. The neuro-probe can have process feasibility and can be formed by conventional process fabrication method(s).

The neuro-probe devices can have the above described characteristics of the carrier and the neuro-probe. The neuro-probe devices using a bio-resorbable glass incorporated with electrode layer and drug delivery mechanism can minimize the tissue damage due to the brain movement without compromising the mechanical strength of the probe for ease of tissue penetration. Due to the flexibility of the electrode layer, tissue damage due to incompatibility to the movement of the brain can be avoided. The probe-tissue post-implantation mismatch can be reduced. Further, the carrier can have the same width dimensions as the neuro-probe so that the penetration area into the tissue is smaller. The scars caused by conventional neuro-probe devices can be minimized or avoided.

Upon successfully penetration of brain tissue, the bio-resorbable glass will react with cerebrospinal fluid (CSF) and degrade within one to two days duration, leaving the electrode layer (e.g. the neuro-probe) behind. The bio-resorbable glass can leave near zero residue.

The neuro-probe devices can be used for neuro probe application (e.g. stimulate neuron, neuron signal transmitter/receiver) and drug delivery.

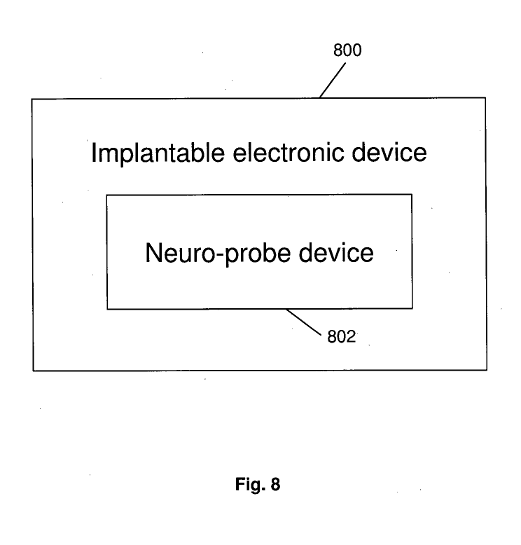

FIG. 8 shows a schematic diagram of an implantable electronic device 800 for neural recording and/or stimulation and/or drug delivery. The implantable electronic device 800 includes at least one neuro-probe device 802. The neuro-probe device 802 can include any one of the neuro-probe devices described above.

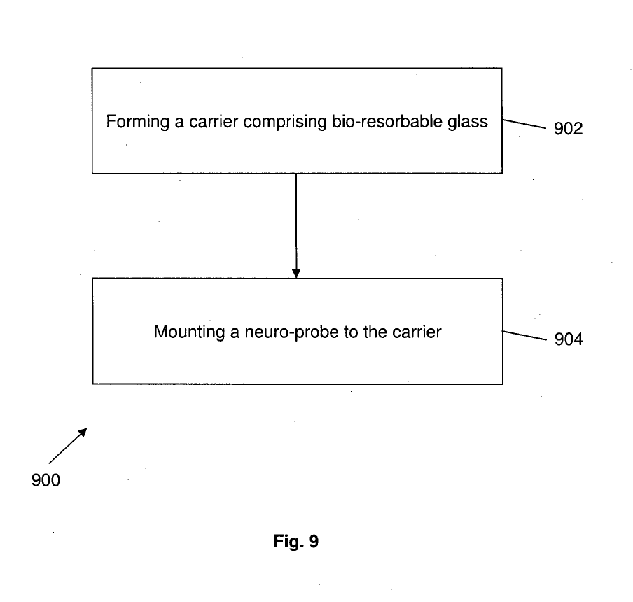

FIG. 9 shows a flowchart 900 of a process of forming a neuro-probe device. At 902, a carrier including bio-resorbable glass is formed. At 904, a neuro-probe is mounted to the carrier.

In one embodiment, forming the carrier may include casting a bio-resorbable glass material having a single degradation rate into a mold having a plurality of patterns of carrier structures, forming a glass wafer including a plurality of carriers, and releasing the glass wafer from the mold and attaching a support wafer to the glass wafer.

In one embodiment, forming the neuro-probe includes disposing a polymer layer above a surface of the carrier facing away from the support wafer, and patterning the polymer layer, disposing an electrode layer above the polymer layer, and patterning the electrode layer, disposing a further polymer layer above the electrode layer, and patterning the further polymer layer to expose portions of the electrode layer.

In one embodiment, the method may further include cutting the glass wafer into individual neuro-probe devices, and removing the support wafer.

In one embodiment, the mold may include a plurality of patterns of recess structures. At least one recess may be formed in the surface of each carrier facing away from the support structure.

In one embodiment, the method may further include disposing drug and/or chemical in the at least one recess of each carrier before the polymer layer is disposed above the surface of the carrier facing away from the support wafer.

In one embodiment, the polymer layer may be patterned to form at least one cavity in the polymer layer. The method may further disposing drug and/or chemical in the at least one cavity of the polymer layer.

In one embodiment, forming the carrier may include casting a plurality of bio-resorbable glass materials having different degradation rates into a mold having a plurality of patterns of carrier structures, and forming a glass wafer including a plurality of carriers. Each carrier may include a plurality of sections. Each section of the carrier may include a bio-resorbable glass material having a different degradation rate.

In one embodiment, forming the carrier may further include releasing the glass wafer from the mold and attaching a support wafer to the glass wafer.

In one embodiment, forming the neuro-probe may include disposing a polymer layer on a surface of the carrier facing away from the support wafer, and patterning the polymer layer, disposing an electrode layer on the polymer layer, and patterning the electrode layer, disposing a further polymer layer on the electrode layer, and patterning the further polymer layer to expose portions of the electrode layer.

In one embodiment, the method may further include cutting the glass wafer into individual neuro-probe devices, and removing the support wafer.

In one embodiment, the mold includes a plurality of patterns of recess structures. A recess may be formed in the surface of each section of the carrier facing away from the support wafer.

In one embodiment, the method may further include disposing drug and/or chemical into each recess of the carrier before the polymer layer is disposed on the surface of the carrier facing away from the support wafer.

In one embodiment, the polymer layer may be patterned to form a plurality of cavities in the polymer layer. Each cavity may be formed above a corresponding section of the carrier.

In one embodiment, the method may further include disposing drug and/or chemical in each cavity of the polymer layer.

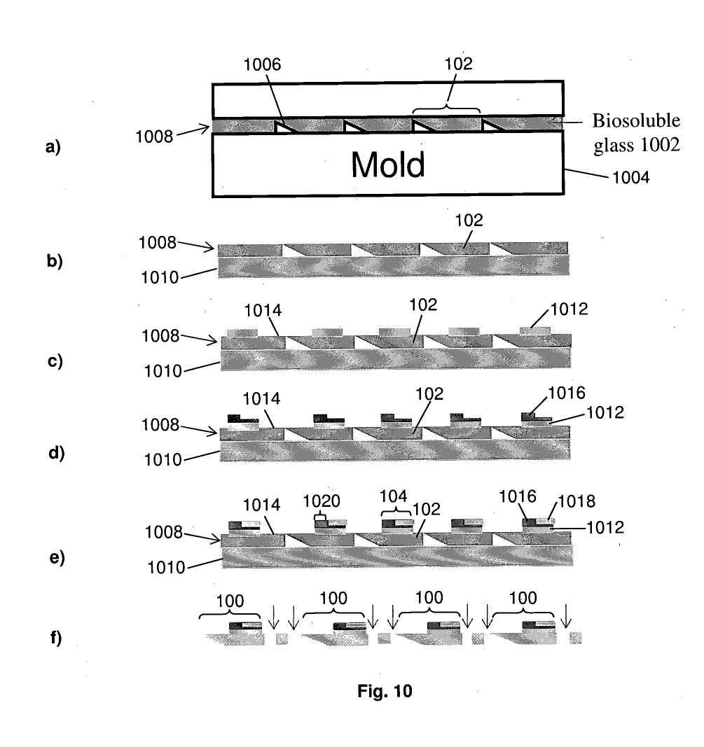

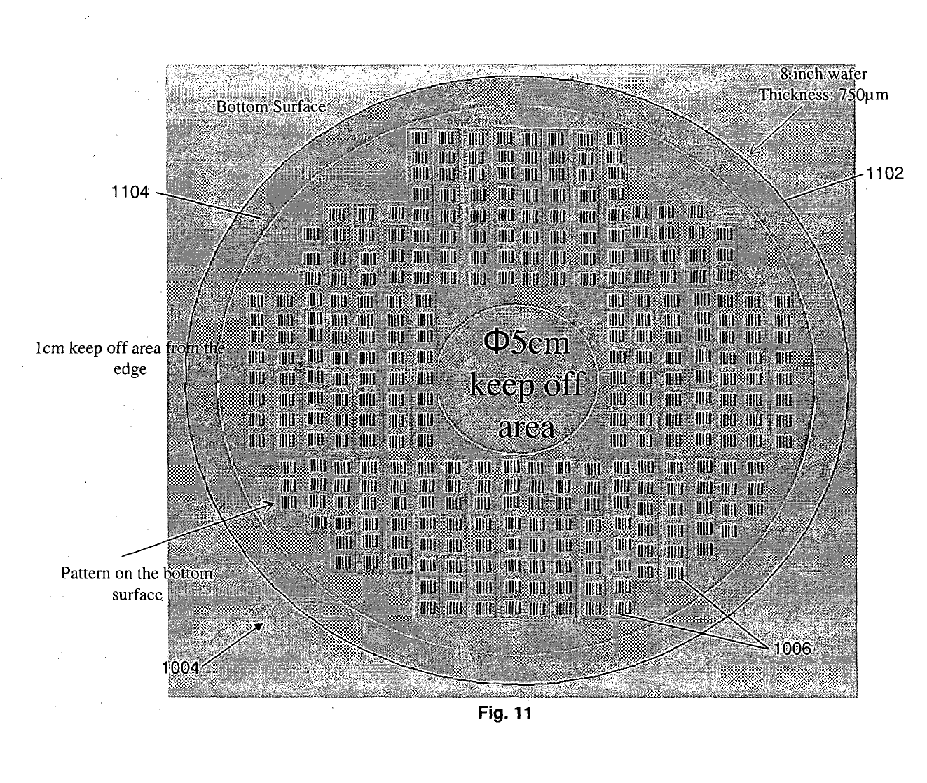

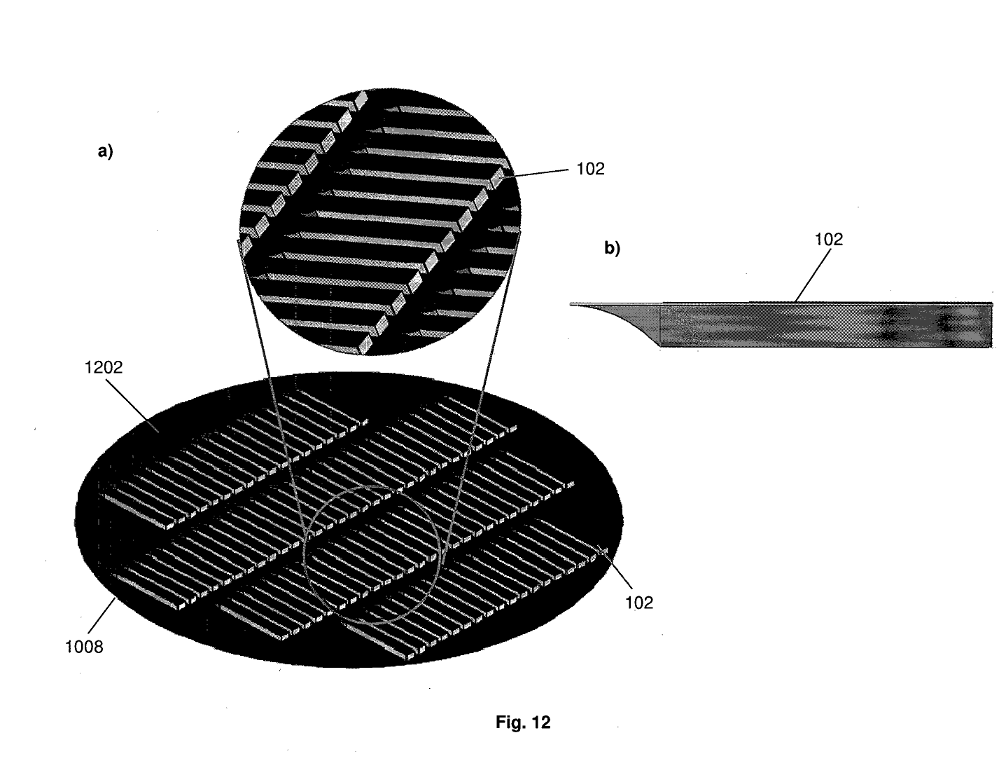

FIGS. 10a-10f show a process of forming the neuro-probe device 100 of FIGS. 2a and 2b according to one embodiment. FIG. 10a shows that a bio-resorbable glass material 1002 having a single degradation rate is casted into a mold 1004 having a plurality of patterns of carrier structures 1006. In one embodiment, the mold 1004 may be an 8 inch wafer 1102 having plurality of patterns of carrier structures 1006 on a bottom surface 1104 of the wafer 1102 as shown in FIG. 11. The raw material of bio-resoluble glass may be melted and casted into the wafer 1102 using glass casting technique. A glass wafer 1008 including a plurality of carriers 102 may be formed. FIG. 12a shows a bottom surface 1202 of the glass wafer 1008 having the plurality of carriers 102. FIG. 12b shows a side view of the carrier 102.

FIG. 10b shows that the glass wafer 1008 is released from the mold 1004 and a support wafer 1010 is attached to the glass wafer 1008. A semiconductor fabrication process may be used subsequently to form the electrode layers (e.g. the neuro-probe 104).

FIG. 10c shows that a polymer layer 1012 is disposed above a surface 1014 of the carrier 102 facing away from the support wafer 1010. The polymer layer 1012 may be disposed above the surface 1014 of the carrier 102 using vapor deposition process. The polymer layer 1012 may be patterned. The polymer layer 1012 may be patterned using a lithography and etching process. The polymer layer 1012 may include but is not limited to parylene, polyimide, polydimethylsiloxane (PDMS) and SU-8.

FIG. 10d shows that an electrode layer 1016 is disposed above the polymer layer 1012 and is patterned. The electrode layer 1016 may include conductive materials. The conductive materials may include but are not limited to gold.

FIG. 10e shows that a further polymer layer 1018 is disposed above the electrode layer 1016. The further polymer layer 1018 may be a passivation layer. The further polymer layer 1018 may include but is not limited to parylene, polyimide, polydimethylsiloxane (PDMS) and SU-8. The further polymer layer 1018 may be patterned to expose portions 1020 of the electrode layer 1016. The exposed portion 1020 of the electrode layer 1016 can be in contact with the neurons for sensing and measurement purposes after the neuro-probe device 100 is inserted into the brain tissue. A plurality of neuro-probes 104 may be formed.

FIG. 10f shows that the glass wafer 1008 is cut into individual neuro-probe devices 100. The glass wafer 1008 may be cut into individual neuro-probe devices 100 using laser cutting technique. The support wafer 1010 may be removed.

In one embodiment, a process similar to the process as described above with reference to FIGS. 10a-10f can be used to form the neuro-probe device 500, whereby a plurality of bio-resorbable glass materials having different degradation rates is casted into a mold having a plurality of patterns of carrier structures. Thus, each carrier 102 formed may include a plurality of sections (e.g. a first section 502a, a second section 502b, a third section 502c and a fourth section 502d of. FIGS. 5a and 5b), and each section of the carrier 102 may include a bio-resorbable glass material having a different degradation rate.

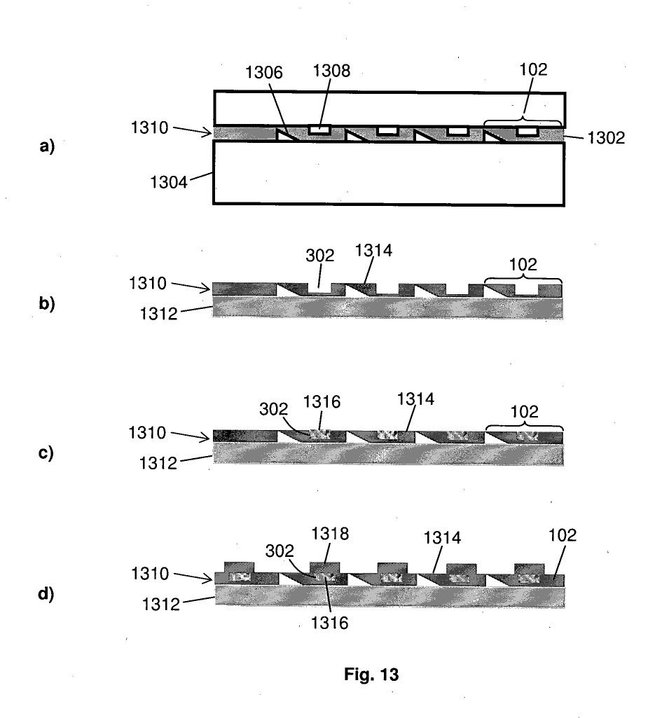

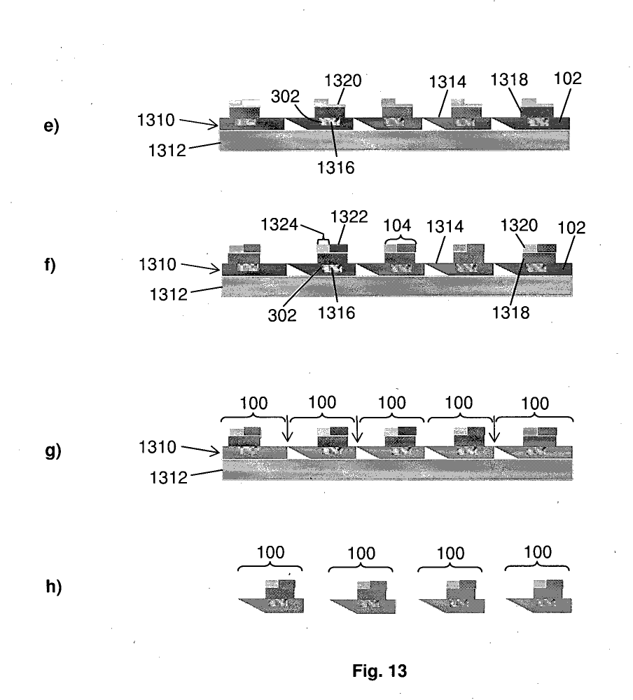

FIGS. 13a-13h show a process of forming the neuro-probe device 100 of FIG. 3 according to one embodiment. FIG. 13a shows that a bio-resorbable glass material 1302 having a single degradation rate is casted into a mold 1304 having a plurality of patterns of carrier structures 1306. The mold 1304 may further include a plurality of recess structures 1308. The bio-resoluble glass material 1302 may be melted and casted into the mold 1304 using glass casting technique. A glass wafer 1310 including a plurality of carriers 102 may be formed.

FIG. 13b shows that the glass wafer 1310 is released from the mold 1304 and a support wafer 1312 is attached to the glass wafer 1310. At least one recess 302 (e.g. one recess) may be formed in a surface 1314 of each carrier 102 facing away from the support wafer 1312.

FIG. 13c shows that drug and/or chemical 1316 is disposed in the recess 302 of each carrier 102. The drug and/or chemical 1316 may include but is not limited to maltose with drug.

A semiconductor fabrication process may be used subsequently to form the the neuro-probe 104. FIG. 13d shows that a polymer layer 1318 is disposed above the surface 1314 of the carrier 102. The polymer layer 1318 may be disposed above the surface 1314 of the carrier 102 using vapor deposition process. The polymer layer 1318 may be patterned. The polymer layer 1318 may be patterned using a lithography and etching process. The polymer layer 1318 may be disposed above the recess 302 of each carrier 102. The polymer layer 1318 may include but is not limited to parylene, polyimide, polydimethylsiloxane (PDMS) and SU-8.

FIG. 13e shows that an electrode layer 1320 is disposed above the polymer layer 1318 and is patterned. The electrode layer 1320 may include conductive materials. The conductive materials may include but are not limited to gold.

FIG. 13f shows that a further polymer layer 1322 is disposed above the electrode layer 1320. The further polymer layer 1322 may be a passivation layer. The further polymer layer 1322 may include but is not limited to parylene, polyimide, polydimethylsiloxane (PDMS) and SU-8. The further polymer layer 1322 may be patterned to expose portions 1324 of the electrode layer 1320. The exposed portion 1324 of the electrode layer 1320 can be in contact with the neurons for sensing and measurement purposes after the neuro-probe device 100 is inserted into the brain tissue. A plurality of neuro-probes 104 may be formed.

FIG. 13g shows that the glass wafer 1310 is cut into individual neuro-probe devices 100. The glass wafer 1310 may be cut into individual neuro-probe devices 100 using laser cutting technique.

FIG. 13h shows that the support wafer 1312 may be removed.

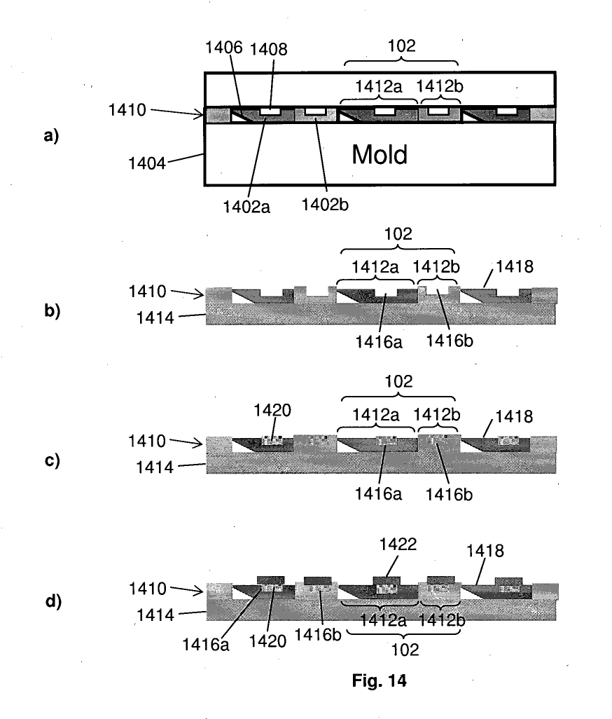

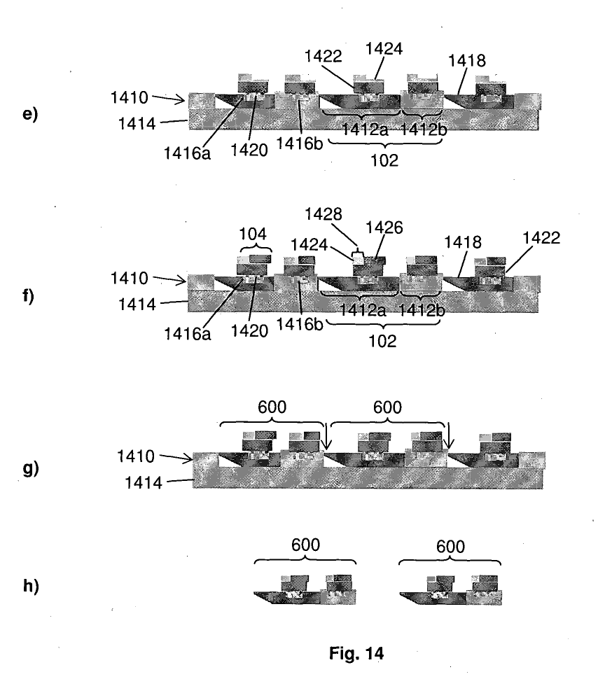

FIGS. 14a-14h show a process of forming an exemplary neuro-probe device 600 according to one embodiment. FIG. 14a shows that a plurality of bio-resorbable glass materials (e.g. a first bio-resorbable glass material 1402a and a second bio-resorbable glass material 1402b) having different degradation rates is casted into a mold 1404 having a plurality of patterns of carrier structures 1406. The mold 1404 may further include a plurality of recess structures 1408. The first bio-resoluble glass material 1402a and the second bio-resorbable glass material 1402b may be melted and casted into the mold 1404 using glass casting technique. For illustration purposes, only two bio-resorbable glass materials are shown. The number of bio-resorbable glass materials used can vary in different embodiments.

A glass wafer 1410 including a plurality of carriers 102 may be formed. Each carrier 102 may include a plurality of sections (e.g. a first section 1412a and a second section 1412b). The first section 1412a and the second section 1412b may include the first bio-resoluble glass material 1402a and the second bio-resorbable glass material 1402b having different degradation rates respectively. For illustration purposes, only two sections are shown. The number of sections can vary in different embodiments. The number of sections may correspond to the number of bio-resorbable glass materials used.

FIG. 14b shows that the glass wafer 1410 is released from the mold 1404 and a support wafer 1414 is attached to the glass wafer 1410. At least one recess (e.g. a first recess 1416a and a second recess 1416b) may be formed in a surface 1418 of each carrier 102 facing away from the support wafer 1414. The first section 1412a and the second section 1412b has the first recess 1416a and the second recess 1416b formed in the surface 1418 of each carrier 102 respectively.

FIG. 14c shows that drug and/or chemical 1420 is disposed in the first recess 1416a and the second recess 1416b of each carrier 102. The drug and/or chemical 1420 may include but is not limited to maltose with drug.

A semiconductor fabrication process may be used subsequently to form the neuro-probe 104. FIG. 14d shows that a polymer layer 1422 is disposed above the surface 1418 of the carrier 102. The polymer layer 1422 may be disposed above the surface 1418 of the carrier 102 using vapor deposition process. The polymer layer 1422 may be patterned. The polymer layer 1422 may be patterned using a lithography and etching process. The polymer layer 1422 may be disposed above the recess 1416a and the second recess 1416b of each carrier 102. The polymer layer 1422 may include but is not limited to parylene, polyimide, polydimethylsiloxane (PDMS) and SU-8.

FIG. 14e shows that an electrode layer 1424 is disposed above the polymer layer 1420 and is patterned. The electrode layer 1424 may include conductive materials. The conductive materials may include but are not limited to gold.

FIG. 14f shows that a further polymer layer 1426 is disposed above the electrode layer 1424. The further polymer layer 1426 may be a passivation layer. The further polymer layer 1426 may include but is not limited to parylene, polyimide, polydimethylsiloxane (PDMS) and SU-8. The further polymer layer 1426 may be patterned to expose portions 1428 of the electrode layer 1424. The exposed portion 1428 of the electrode layer 1424 can be in contact with the neurons for sensing and measurement purposes after the neuro-probe device 600 is inserted into the brain tissue. A plurality of neuro-probes 104 may be formed.

FIG. 14g shows that the glass wafer 1410 is cut into individual neuro-probe devices 600. The glass wafer 1410 may be cut into individual neuro-probe devices 600 using laser cutting technique.

FIG. 14h shows that the support wafer 1414 may be removed.

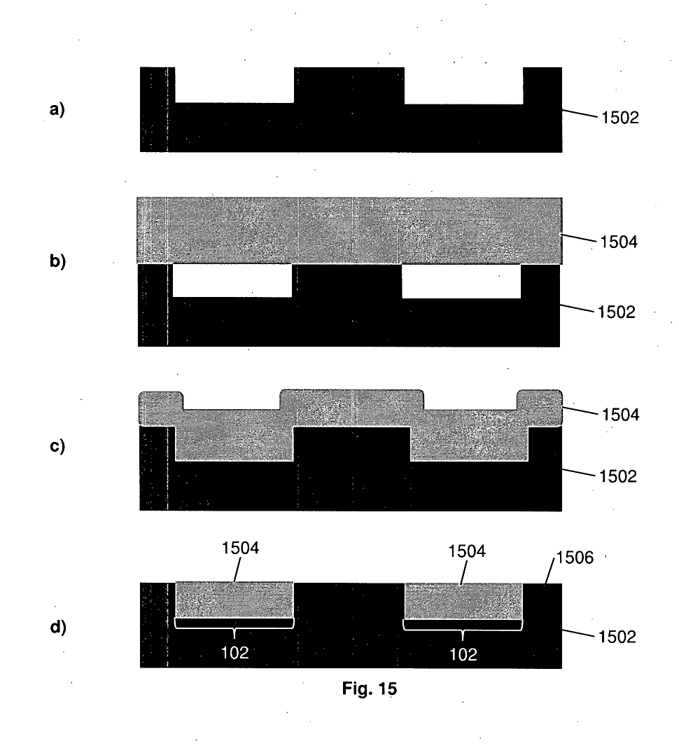

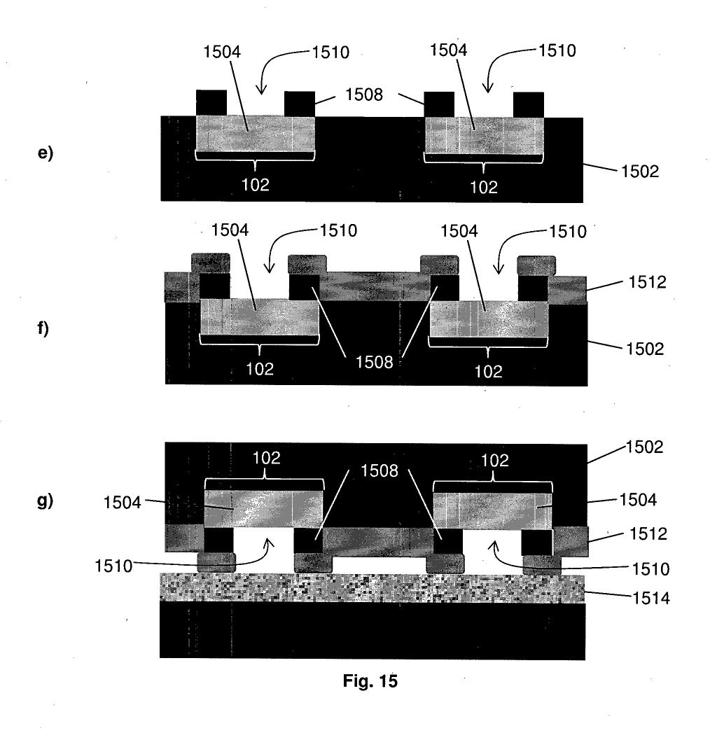

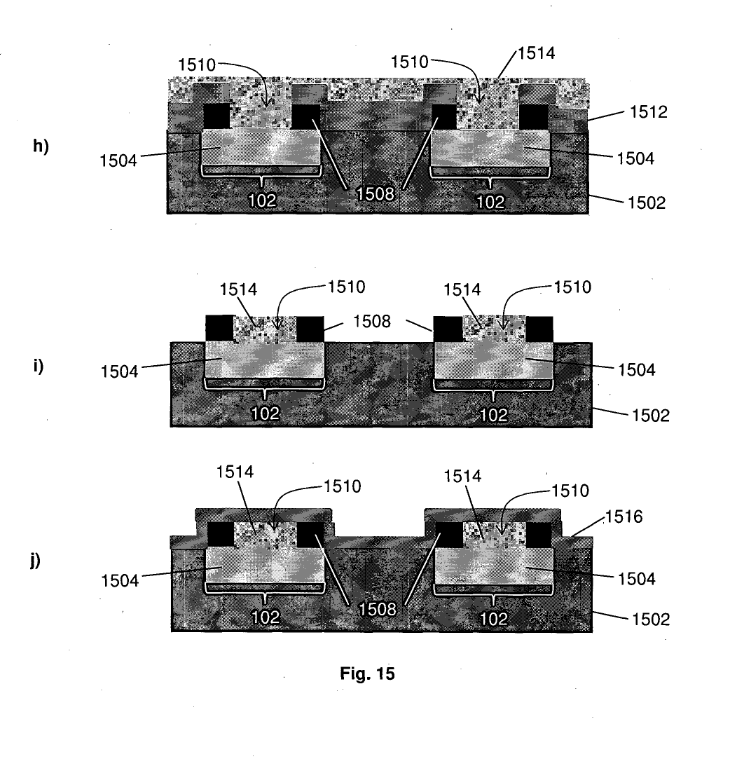

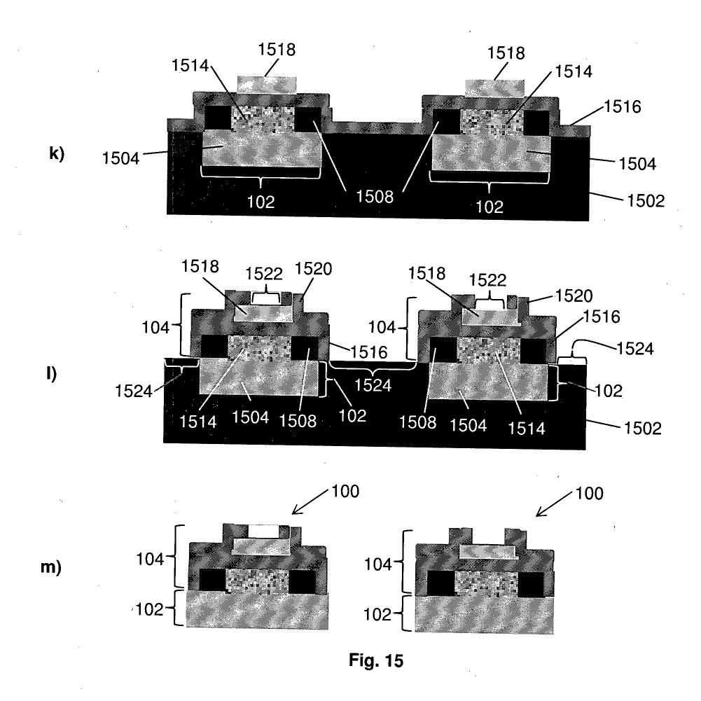

FIGS. 15a-15m show a process of forming an exemplary neuro-probe device 100 of FIG. 4 according to one embodiment. FIG. 15a shows a mold 1502 used for forming the neuro-probe device 100. The mold 1502 may include but is not limited to silicon.

FIG. 15b shows that a bio-resorbable glass (e.g. bioglass) 1504 is disposed above the mold 1502. The bio-resorbable glass 1504 may be disposed above the mold 1502 by anodic bonding at 1000V and at 400° C. (which may be based on requirements for Pyrex 7740 glass). An etch stop layer (not shown) may be added to the mold 1502 before the bio-resorbable glass 1504 is disposed above the mold 1502. The purpose of the etch stop layer is to protect the device during the last release step (deep reactive ion etching (DRIE)) in FIG. 15m. The etch stop layer may include but is not limited to aluminum and silicon dioxide. The etch stop layer may have a thickness of about 100 nm.

FIG. 15c shows that the bio-resorbable glass 1504 is melted. The bio-resorbable glass 1504 may be melted at about 750° C. and for about 7 hours (which may be based on requirements for Pyrex 7740 glass). Generally, a melting temperature for e.g. a bio-resorbable glass is about 500° C. Process optimization may be required if the bio-resorbable glass is melted at about 500° C. The melted bio-resorbable glass 1504 may be casted into the mold 1502.

FIG. 15d shows that the bio-resorbable glass 1504 is planarized to a surface 1506 of the mold 1502. Lapping may be carried out to planarize the bio-resorbable glass 1504. The carrier 102 is formed.

FIG. 15e shows that a negative resist layer 1508 is disposed above the mold 1502 and the carrier 102. The negative resist layer 1508 may be patterned and cured to form a cavity 1510 above the carrier 102. The negative resist layer 1508 may be biocompatible. The negative resist layer 1508 may include but is not limited to SU-8. The cavity 1510 may be used for drug retention.

FIG. 15f shows that a sacrificial layer 1512 is disposed above the mold 1502 and the negative resist layer 1508. The sacrificial layer 1512 may be cured at about 120° C. for about 2 minutes. The sacrificial layer 1512 may include but is not limited to photodefinable polydimethylsiloxane (PDMS). The sacrificial layer 1512 may be provided for drug filling in the cavity 1510.

FIG. 15g shows that the sacrificial layer 1512 is placed in contact with solid maltose candy mixed with drug 1514. FIG. 15h shows that the cavity 1510 may be filled with the maltose candy mixed with drug 1514. The solid maltose candy mixed with drug 1514 may be heated at about 95° C. for about 1 minute. The cavity 1510 may then be filled with the maltose candy mixed with drug 1514 by micro molding in capillary method.

FIG. 15i shows that the sacrificial layer 1512 is removed, leaving the hardened maltose candy mixed with drug 1514 in the cavity 1510.

FIG. 15j shows that a polymer layer 1516 is disposed above the mold 1502, the negative resist layer 1508 and the maltose candy mixed with drug 1514 in the cavity 1510. The polymer layer 1516 may be used to encase the maltose candy mixed with drug 1514 in the cavity 1510. The polymer layer 1516 may include but is not limited to parylene.

FIG. 15k shows that an electrode layer 1518 is disposed above the polymer layer 1516 and is patterned. The electrode layer 1518 may include but is not limited to gold.

FIG. 15l shows that a further polymer layer 1520 is disposed above the polymer layer 1516 and the electrode layer 1518. The further polymer layer 1520 and the polymer layer 1516 are patterned to expose portions 1522 of the electrode layer 1518 and portions 1524 of the mold 1502. The neuro-probe 104 is formed.

FIG. 15m shows that the mold 1502 is removed to form the individual neuro-probes 100 of FIG. 4. The mold 1502 may be removed using deep reactive ion etching (DRIE).

In one embodiment, a process similar to the process as described above with reference to FIGS. 15a-15m can be used to form the neuro-probe device 700 of FIG. 7, whereby a plurality of bio-resorbable glass materials having different degradation rates may be casted into the mold 1502. The cavity 1510 may be formed above each of a plurality of sections of the carrier 102 for drug retention.

The above described processes of forming the neuro-probe device are simple fabrication processes and can be easy to handle. Standard microfabrication processes can be used. Biocompatible materials are used. Microelectromechanical systems (MEMS) fabrication can be used using new biocompatible materials. No spurious contamination can be achieved due to removal of sacrificial material. The above described processes can provide lithographic definition and relative positioning of the microfabricated flexible and stiff portions of the neuro-probe device. Batch fabrication (e.g. on a wafer level) is possible using the above described processes.

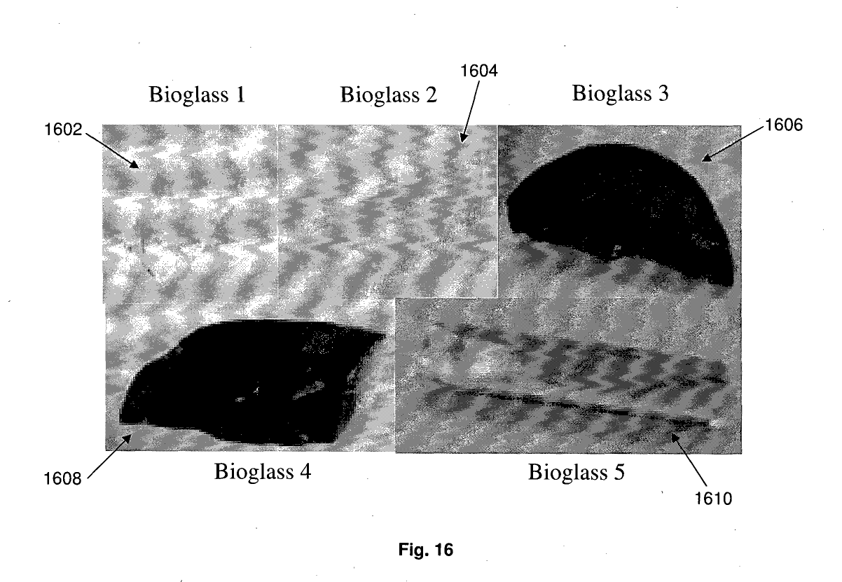

Preliminary degradation tests were performed in deionized (DI) water and simulated brain fluid using bio-resoluble glass samples. The experiment was carried out to evaluate the degradation rates of the different bio-resorbable (bio-resoluble) glass materials. FIG. 16 shows the five bio-resoluble glass materials used in the experiment. The first bio-resoluble glass 1602 is fluoride phosphate based soluble glass. The second bio-resoluble glass 1604 is zinc phosphate based soluble glass. The third bio-resoluble glass 1606 is copper phosphate based soluble glass (type 1). The fourth bio-resoluble glass 1608 is copper phosphate based soluble glass (type 2). The fifth bio-resoluble glass 1610 is GL0811 bioactive glass.

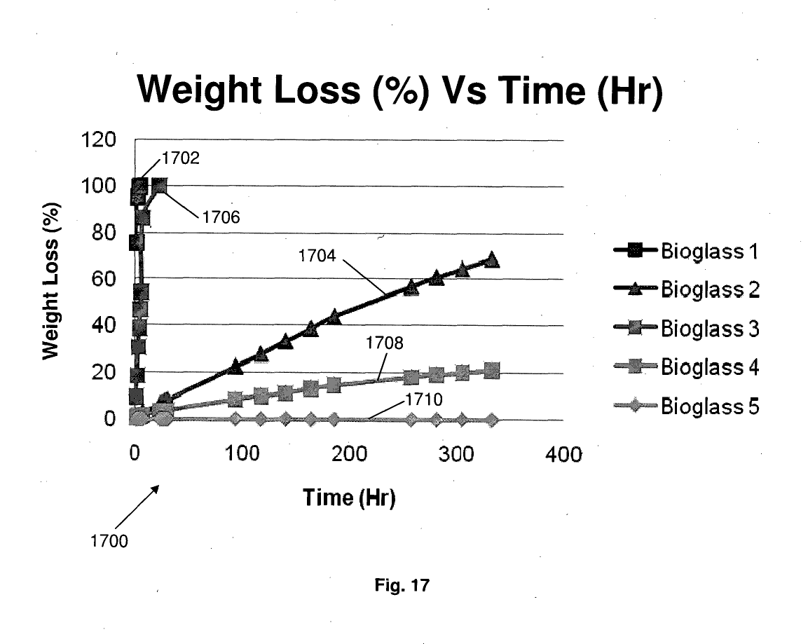

The samples (i.e. five bio-resoluble glass materials 1602, 1604, 1606, 1608, 1610) were first placed in the DI water under ambient condition. FIG. 17 shows a graph 1700 of weight loss (%) plotted against time (hour). Graph 1700 shows the dissolution rate of the samples in DI water. Plot 1702 shows the dissolution rate of the first bio-resoluble glass 1602. Plot 1704 shows the dissolution rate of the second bio-resoluble glass 1604. Plot 1706 shows the dissolution rate of the third bio-resoluble glass 1606. Plot 1708 shows the dissolution rate of the fourth bio-resoluble glass 1608. Plot 1710 shows the dissolution rate of the fifth bio-resoluble glass 1610.

Graph 1700 shows that the first bio-resoluble glass 1602 has the fastest degradation rate of about 4 hours and the third bio-resoluble glass 1606 has the second fastest degradation rate of about 1 day. The second bio-resoluble glass 1604 has the third fastest degradation rate followed by the fourth bio-resoluble glass 1608. The fifth bio-resoluble glass 1610 has the slowest degradation rate.

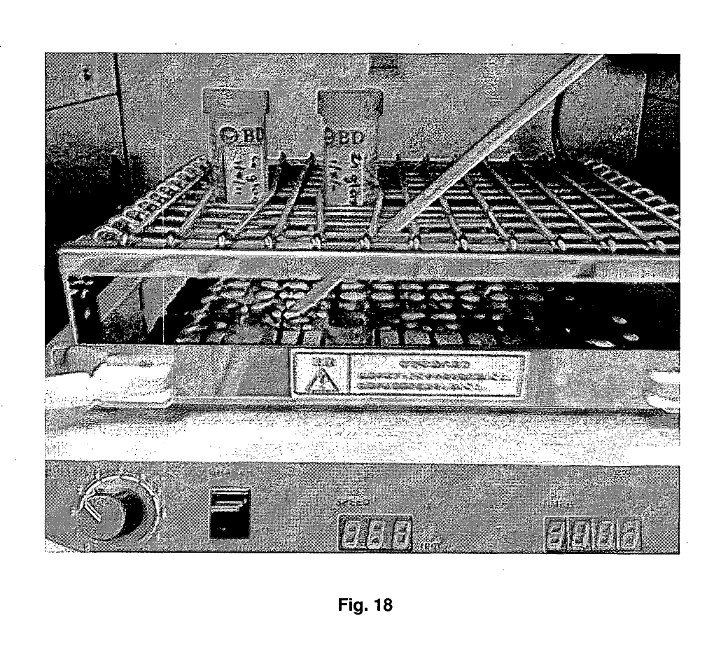

A further degradation test in simulated brain fluid was performed for the second bio-resoluble glass 1604 and the third bio-resoluble glass 1606. The experiment was carried out at about 37° C. which corresponds to the body temperature and under a rotation speed of about 60 rpm. FIG. 18 shows the experimental setup.

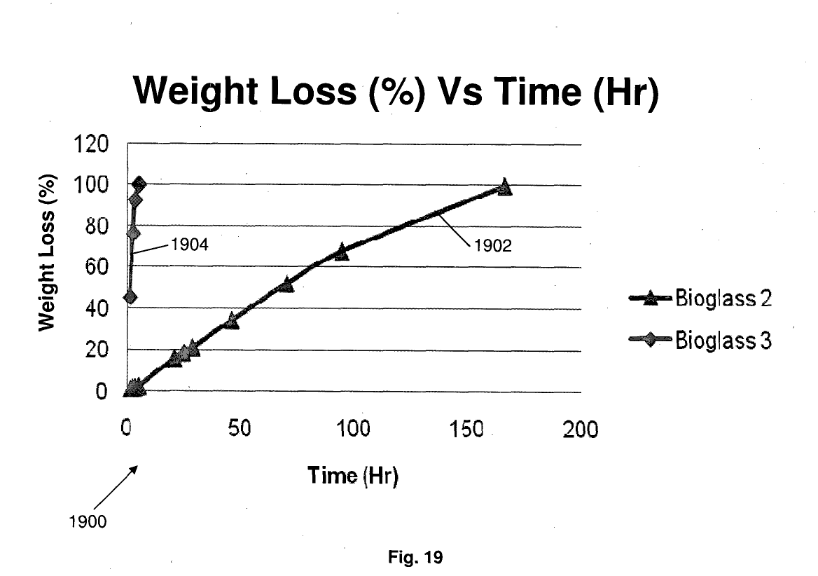

FIG. 19 shows a graph 1900 of weight loss (%) plotted against time (hour). Graph 1900 shows the dissolution rate of the second bio-resoluble glass 1604 and the third bio-resoluble glass 1606 in simulated brain fluid. Plot 1902 shows the dissolution rate of the second bio-resoluble glass 1604. Plot 1904 shows the dissolution rate of the third bio-resoluble glass 1606.

It can be observed from graph 1900 that the third bio-resoluble glass 1606 has completely degraded in the simulated brain fluid within a duration of about 4 hours, and the second bio-resoluble glass 1604 has completely degraded after about 7 days.

An ex vivo fluoroscope test was also performed on a pig specimen. This test was carried out to evaluate the radiopacity of the five bio-resoluble glass samples 1602, 1604, 1606, 1608, 1610. The first bio-resoluble glass 1602, the second bio-resoluble glass 1604, the third bio-resoluble glass 1606, the fourth bio-resoluble glass 1608 and the fifth bio-resoluble glass 1610 are placed on the surface of the pig's skin and a medical fluoroscope system is being employed.

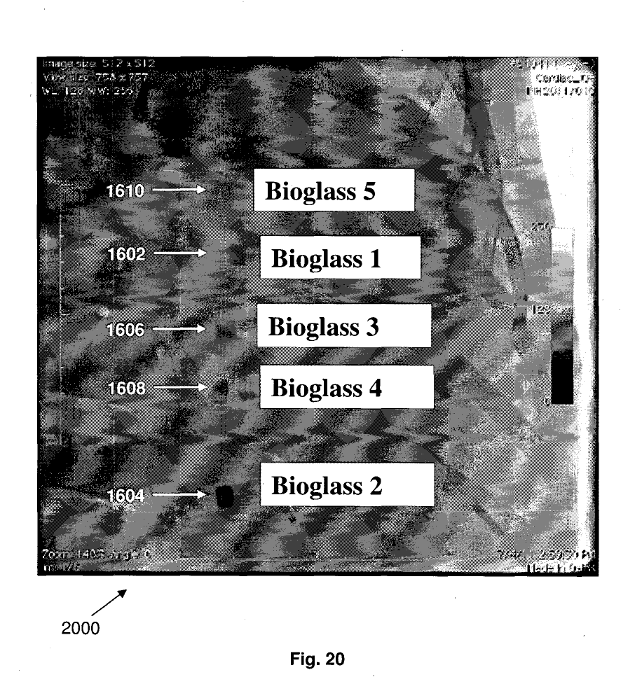

FIG. 20 shows a fluoroscope image 2000 of the first bio-resoluble glass 1602, the second bio-resoluble glass 1604, the third bio-resoluble glass 1606, the fourth bio-resoluble glass 1608 and the fifth bio-resoluble glass 1610 on the pig's skin. It can be observed from the fluoroscope image 2000 that the second bio-resoluble glass 1604 has the highest radiopacity.

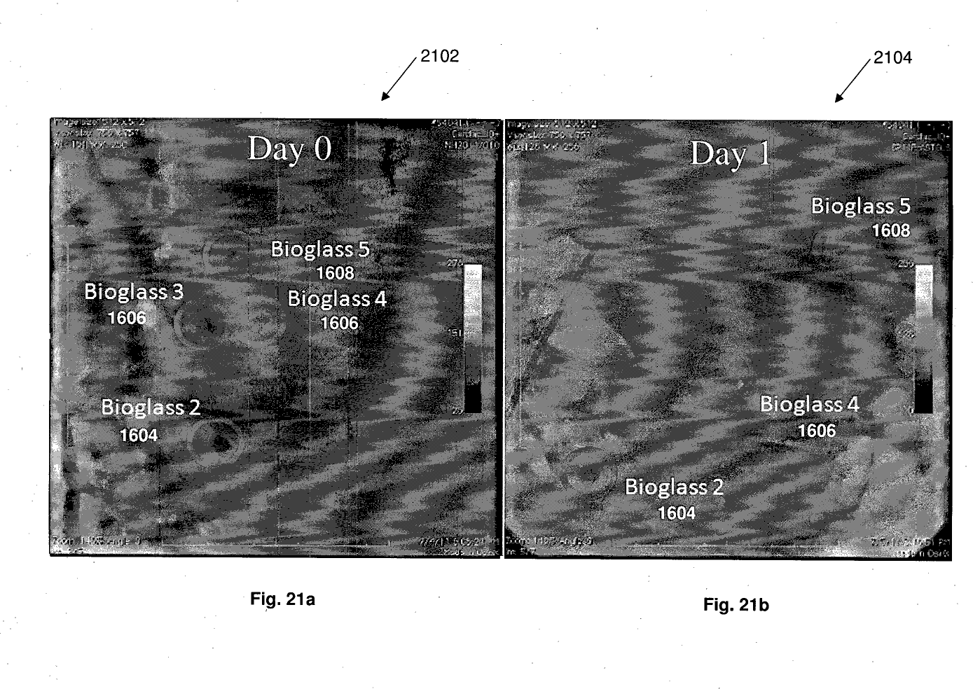

An in vivo degradation test was performed on the pig specimen to determine the complete degradation period of the five glass samples 1602, 1604, 1606, 1608, 1610. A fluoroscope check was performed about 4 hours after implantation of the first bio-resoluble glass 1602, the second bio-resoluble glass 1604, the third bio-resoluble glass 1606, the fourth bio-resoluble glass 1608 and the fifth bio-resoluble glass 1610. FIG. 21a shows the fluoroscope image 2102 about 4 hours after implantation. It can be observed from the fluoroscope image 2102 that the first bio-resoluble glass 1602 has completely degraded.

Another fluoroscope check was performed on the next day of the implantation. FIG. 21b shows the fluoroscope image 2104 on the next day of the implantation. It can be observed from the fluoroscope image 2104 that the third bio-resoluble glass 1606 has completely degraded. These results correspond to the results observed in the preliminary degradation test (i.e. shown in graph 1700 of FIG. 17).

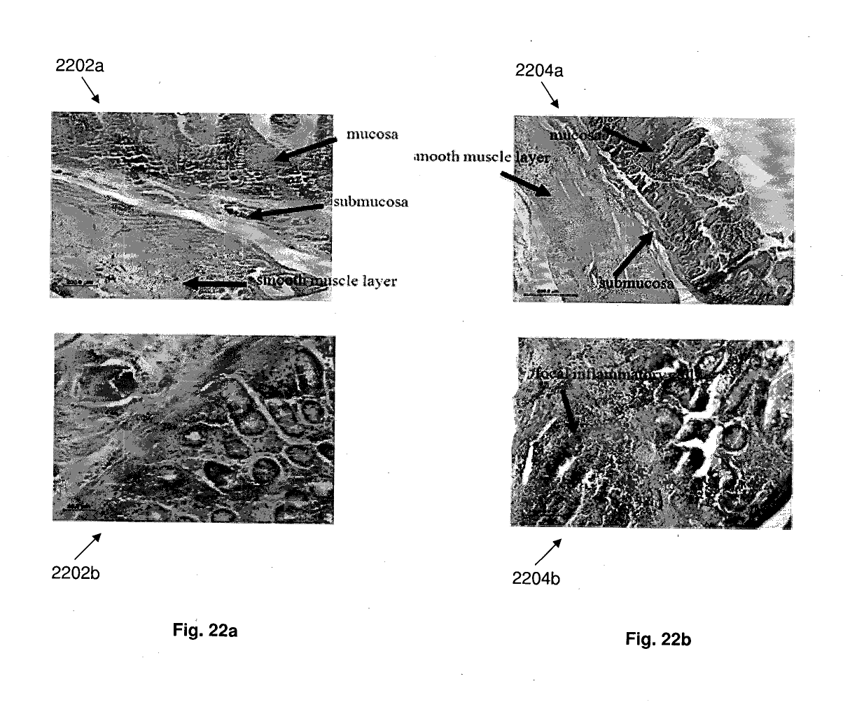

The specimen was kept and euthanized after a month. Tissue segments of the implanted second bio-resoluble glass 1604 and the implanted third bio-resoluble glass 1606 were harvested and sent for histology analyses. FIG. 22a shows microscope images 2202a, 2202b of the tissue segment of the implanted second bio-resoluble glass 1604. FIG. 22b shows microscope images 2204a, 2204b of the tissue segment of the implanted third bio-resoluble glass 1606. It can be observed from the microscope images 2202a, 2202b, 2204a, 2204b that there was minimal inflammation with focal mononuclear cells infiltration in the tissue segment of the third bio-resoluble glass implanted site and there was no significant inflammation detected in the tissue segment of the second bio-resoluble glass implanted site, except for the normal presence of lymphocytes, plasma cells in the lamina propria in the mucosa layer. The inflammatory infiltration was mostly lymphatic in nature and highly concentrated immediately, but selectively focal at the underlying submucosa segment basal to the mucosa layer. However, no foreign body reaction or granuloma was noticeable in both the second bio-resoluble glass implanted site and the third bio-resoluble glass implanted site. Furthermore, the supporting smooth muscle layers in the second bio-resoluble glass implanted site and the third bio-resoluble glass implanted site were sparse of inflammation with preserved muscular architecture.

The second bio-resoluble glass 1604 evoked significantly less inflammatory response from the host in the current animal tested. Both the second bio-resoluble glass 1604 and the third bio-resoluble glass 1606 do not induce foreign body reaction. No granuloma was observed and the preserved villi in the mucosa suggest good overall biocompatibility of both the second bio-resoluble glass 1604 and the third bio-resoluble glass 1606.

Further, biodegradation tests were also conducted in cerebrospinal fluid (CSF) and in DI water for three samples. The first sample (also referred as “Sample 1”) has a composition (by weight) of 80% phosphorous pentoxide (P₂O₅), 18% sodium oxide (Na₂O) and 2% barium oxide (BaO). The second sample (also referred as “Sample 2”) has a composition (by weight) of 87% boron trioxide (B₂O₃), 2% barium oxide (BaO) and 11% potassium oxide (K₂O). The third sample (also referred as “Sample 3”) has a composition (by weight) of 85% boron trioxide (B₂O₃), 2% barium oxide (BaO), 11% potassium oxide (K₂O) and 2% aluminum oxide (AL₂O₃).

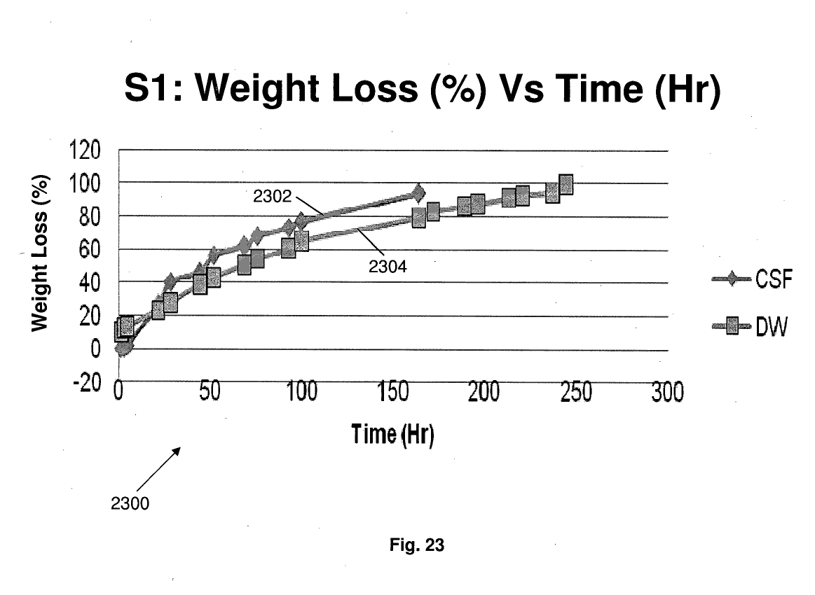

FIG. 23 shows a graph 2300 of weight loss (%) plotted against time (hour) for Sample 1. Plot 2302 shows the dissolution rate of Sample 1 in CSF fluid. Plot 2304 shows the dissolution rate of Sample 1 in DI water. It can be observed from graph 2300 that Sample 1 has a faster degradation rate in CSF fluid than in DI water. Sample 1 has degraded within 7 days in CSF fluid.

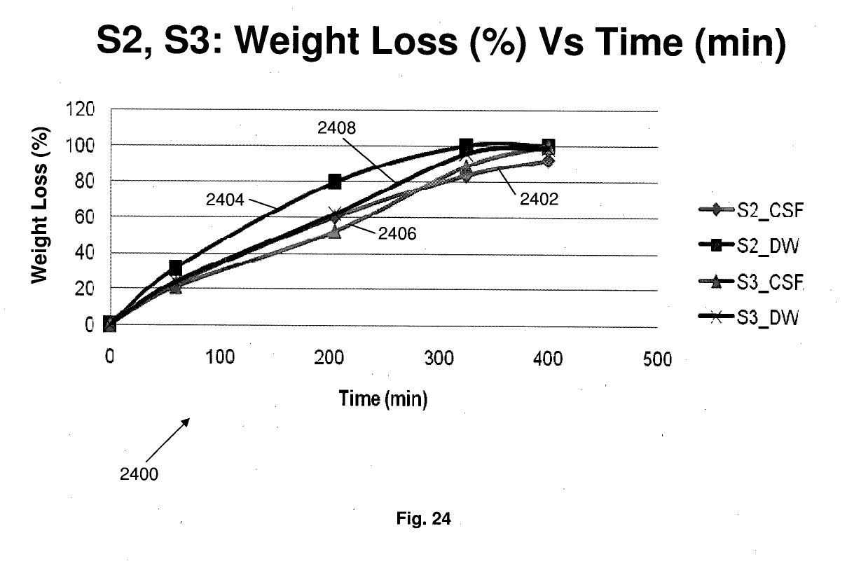

FIG. 24 shows a graph 2400 of weight loss (%) plotted against time (minute) for Sample 2 and Sample 3. Plot 2402 shows the dissolution rate of Sample 2 in CSF fluid. Plot 2404 shows the dissolution rate of Sample 2 in DI water. Plot 2406 shows the dissolution rate of Sample 3 in CSF fluid. Plot 2408 shows the dissolution rate of Sample 3 in DI water. It can be observed from graph 2400 that both Sample 2 and Sample 3 have degraded in CSF fluid and DI water within 7 hours.

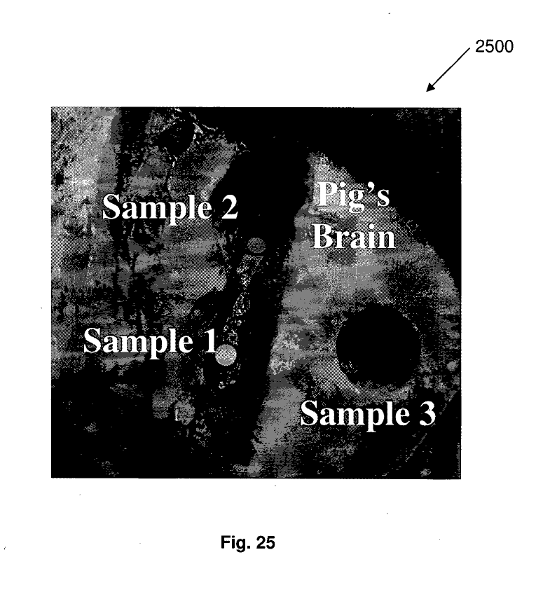

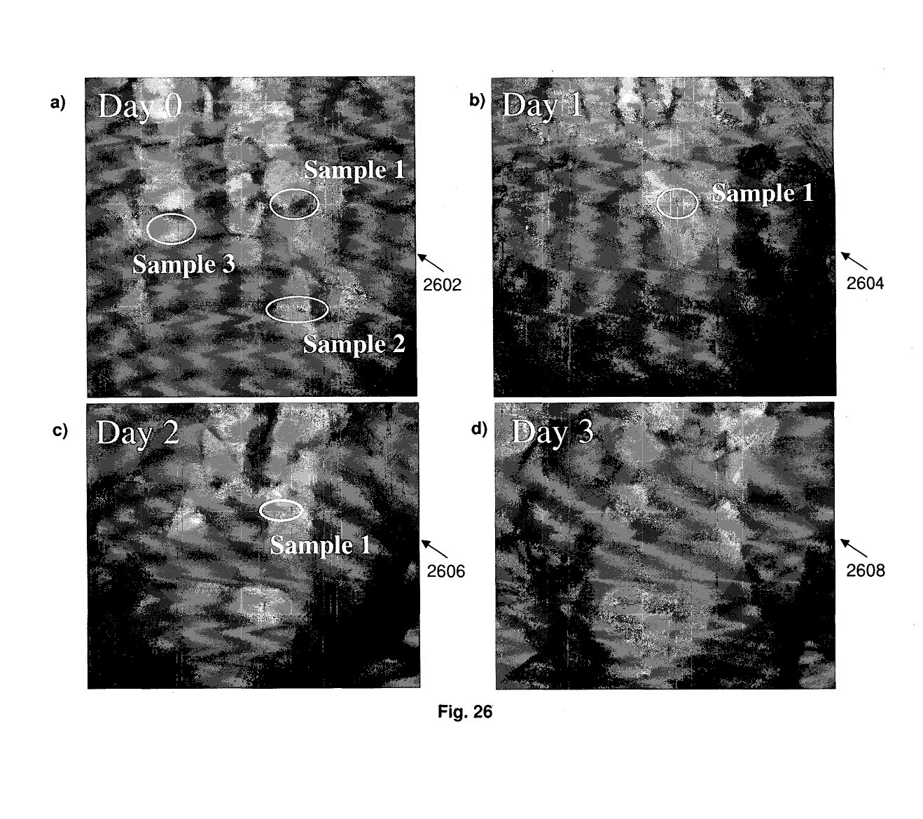

An in vivo degradation test was performed on the pig specimen (e.g. pig's brain) to determine the complete degradation period of Samples 1-3 as shown in the picture 2500 of FIG. 25. FIG. 26a shows a fluoroscope image 2602 after implantation of Samples 1-3. FIG. 26b shows a fluoroscope image 2604 one day after implantation of Samples 1-3. It can be observed from the fluoroscope image 2604 that Sample 2 and Sample 3 have completely degraded. FIG. 26c shows a fluoroscope image 2606 two days after implantation of Samples 1-3. It can be observed from the fluoroscope image 2606 that the radiopacity of Sample 1 has reduced and the size of Sample 1 has decreased. FIG. 26d shows a fluoroscope image 2608 three days after implantation of Samples 1-3. It can be observed from the fluoroscope image 2608 that Sample 1 has completely degraded.

While embodiments of the invention have been particularly shown and described with reference to specific embodiments, it should be understood by those skilled in the art that various changes in form and detail may be made therein without departing from the spirit and scope of the invention as defined by the appended claims. The scope of the invention is thus indicated by the appended claims and all changes which come within the meaning and range of equivalency of the claims are therefore intended to be embraced.

1. A neuro-probe device configured for penetration into a biological tissue, comprising:
a carrier comprising bio-resorbable glass; and
a neuro-probe mounted on the carrier;
wherein the carrier is substantially rigid so as to provide strength to the neuro-probe to penetrate the biological tissue and wherein the bio-resorbable glass is configured to degrade after penetration to leave the neuro-probe behind.

2. The neuro-probe device of claim 1,
wherein the neuro-probe comprises a polymer layer disposed above the carrier and an electrode layer disposed above the polymer layer.

3. The neuro-probe device of claim 1,
wherein the bio-resorbable glass material has a single degradation rate.

4. The neuro-probe device of claim 1,
wherein the carrier comprises at least one recess formed in a surface of the carrier facing the polymer layer.

5. The neuro-probe device of claim 4,
further comprising drug and/or chemical disposed in the at least one recess of the carrier.

6. The neuro-probe device of claim 1,
wherein the polymer layer comprises at least one cavity.

7. The neuro-probe device of claim 6,
further comprising drug and/or chemical disposed in the at least one cavity of the polymer layer.

8. The neuro-probe device of claim 1,
wherein the carrier comprises a plurality of sections, wherein the sections of the carrier comprise different bio-resorbable glass materials.

9. The neuro-probe device of claim 8,
wherein the carrier comprises a recess formed in a surface of each section of the carrier facing the polymer layer.

10. The neuro-probe device of claim 9,
further comprising drug and/or chemical disposed in each recess of the carrier.

11. The neuro-probe device of claim 8,
wherein the polymer layer comprises a plurality of cavities, wherein each cavity of the polymer layer is formed above a corresponding section of the carrier.

12. The neuro-probe device of claim 11,
further comprising drug and/or chemical disposed in each cavity of the polymer layer.

13. The neuro-probe device of claim 1,
wherein the carrier comprises a planar portion having a first surface and a second surface facing away from the first surface.

14. The neuro-probe device of claim 13,
wherein two opposite sides of the first surface and two opposite sides of the second surface converge to form a tip.

15. The neuro-probe device of claim 1,
wherein the bio-resorbable glass comprises any one of a group consisting of fluoride phosphate based soluble glass, zinc phosphate based soluble glass, copper phosphate based soluble glass, boron trioxide based soluble glass, and bioactive glass.

16. (canceled)

17. The neuro-probe device of claim 2,
wherein the polymer layer comprises any one of a group consisting of parylene, polyimide, polydimethylsiloxane (PDMS) and SU-8.

18. (canceled)

19. The neuro-probe device of claim 1,
wherein the neuro-probe is a flexible neuro-probe that is configured to be implantable into a biological tissue.

20. An implantable electronic device for neural recording and/or stimulation and/or drug delivery, the implantable electronic device comprising:
at least one neuro-probe device, each neuro-probe device configured for penetration into a biological tissue, and each neuro-probe device comprising:
a carrier comprising bio-resorbable glass; and
a neuro-probe mounted on the carrier;
wherein each carrier is substantially rigid so as to provide strength to the neuro-probe to penetrate the biological tissue and wherein the bio-resorbable glass is configured to degrade after penetration to leave the neuro-probe behind.

21. A method of forming a neuro-probe device configured for penetration into a biological tissue, the method comprising:
forming a carrier comprising bio-resorbable glass; and
mounting a neuro-probe to the carrier;
wherein the carrier is formed such that the carrier is substantially rigid so as to provide strength to the neuro-probe to penetrate the biological tissue and wherein the bio-resorbable glass is configured to degrade after penetration to leave the neuro-probe behind.

22. The method of claim 21,
wherein forming the carrier comprises:
casting a bio-resorbable glass material having a single degradation rate into a mold having a plurality of patterns of carrier structures;
forming a glass wafer comprising a plurality of carriers;
releasing the glass wafer from the mold and attaching a support wafer to the glass wafer.

23-38. (canceled)
